SEQUENCE LISTING (11 sequences)

SEQ ID NO: 1 - DNA, 2005 bp, Homo sapiens
ttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagttct

SEQ ID NO: 2 - protein, 382 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKSSLWEAPRGQNMLDGGAPFYTTYRTADGEFMAVGAIEPQFYELLIKGLGLKSDELPNQMSMDDWPEMKKKFADVFAKKTKAEWCQIFDGTDACVTPVLTFEEVVHHDHNKERGSFITSEEQDVSPRPAPLLLNTPAIPSFKRDPFIGEHTEEILEEFGFSREEIYQLNSDKIIESNKVKASL

SEQ ID NO: 3 - DNA, 1146 bp, Homo sapiens
  CDS (1)...(1146)
atggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctc

SEQ ID NO: 4 - DNA, 2069 bp, Homo sapiens
  CDS (90)...(1235)
gggcgccgggattgggagggcttcttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatctcaggaccccccaaaaaaaaaaaaaaaaaaaaaaaaaaaaa

SEQ ID NO: 5 - protein, 382 aa, Homo sapiens; identical to SEQ ID NO: 2

SEQ ID NO: 6 - DNA, 3654 bp, Homo sapiens
  CDS (90)...(1271)
gggcgccgggattgggagggcttcttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggctggtagcaagttctggatcttatacccaacacacagcaacatccagaaataaagatctcaggaccccccagcaagtcgttttgtgtctccttggactgagttaagttacaagcctttcttatacctgtctttgacaaagaagacgggattgtctttacataaaaccagcctgctcctggagcttccctggactcaacttcctaaaggcatgtgaggaaggggtagattccacaatctaatccgggtgccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcattgtgtttgtgtgttcaaatttttaaatattctgagatactcttgtgaggtcactctaatgccctgggtgccttggcacagttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggtatggtggtgtgtgcctgtagtcctatctactcaagggtggggcaggaggatcacttgagcccaggaatttgaggccacagtgaattaggattgcaccactgcactctagcccaggcaacagaacaagaacctgtctctaaataaataaataaaaataataataataaaaaagatgttttccctacaaaaaagacttttcatttgaactcggtccagcaaggaaaatataacccactcgaagtctttaaaacagaggaaatttaatataaagaattccactggtgacgaaagagcagagaagcccagaagatagtgaggcaaccctgataggaacataactaggaagccaagaccactcctatggttgcaggggtgatgggaaagctggtgtacttggacccagaagccaaagttgctgcacccaccttggagacatagacactggcagtaatacctcagggagaagaaagaaatctagggaaatatcctggcttctttccttctctctctcccctagtcttcctaccagtgtctcccattagccaaatctacctagaagccagaaaacaagggaaccctggaaatgtagccccataagataaagagcaccaaaggaaatagatctgagcagacaggcagcacaaaatgcagtgtgtatggtttattcactcagtaattcctttagcaaatgtttattgaggatctactaggtgccaggtatcatgatacttgctggggataccataatgaacaaaacagacctgttctccgctcttgaggaaatcaaagacaaacacaggatatggaataaacccagaattatctcattgtaaaatgtgttaagtaccacgaggagaaatatcagggccatctgacacagctaatgatttgaagaagggtgtgacctgccaccattttaaatctagttatttcactcctgagctgtgtgtgtggaaaacttgtagtaaaaaatagaatgtctatatttataaaaagtttatgaaaagatatcaatttatttacattttgacaaactctatgtaataaggctttattactcacggccatgtgtgtgatcatgtgtaatagcatgtgtgtatgagagagagaagccatatgtaattatgtgtaataacgtctgtgagagagaagccatgtgtgtgatcatgtaaaataacgtgtgtgagagaagccatgtgtgtgatcgtgtaaaataacgtgtgtgagaagccgtgtgtgatgtgt

SEQ ID NO: 7 - protein, 394 aa, Homo sapiens [sequence omitted]

SEQ ID NO: 8 - DNA, 3023 bp, Homo sapiens
  CDS (90)...(683)
gggcgccgggattgggagggcttcttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatctcaggaccccccagcaagtcgttttgtgtctccttggactgagttaagttacaagcctttcttatacctgtctttgacaaagaagacgggattgtctttacataaaaccagcctgctcctggagcttccctggactcaacttcctaaaggcatgtgaggaaggggtagattccacaatctaatccgggtgccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcattgtgtttgtgtgttcaaatttttaaatattctgagatactcttgtgaggtcactctaatgccctgggtgccttggcacagttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggaaaaaaaaaaaaaaaaaaaaaaaaaaa

SEQ ID NO: 9 - protein, 198 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGGRNSIFKFFSVENSEIESVGSTSRTEHVGWWSTFLYDLQDSRWGIHGCWSNRTPVLRAADQRTWTKV

SEQ ID NO: 10 - DNA, 2005 bp, Homo sapiens
  CDS (66)...(1211)
ttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatct

SEQ ID NO: 11 - protein, 382 aa, Homo sapiens; identical to SEQ ID NO: 2

(19) United States
(12) Patent Application Publication
(10) Pub. No.: US 2009/0148834 A1
(43) Pub. Date: Jun. 11, 2009

(54) Methods of use of alpha-methylacyl-CoA racemase in hormone refractory and metastatic prostate cancers

(75) Inventors: Jennifer Richardson, Boston, MA (US); John Monahan, Walpole, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/820,705

(22) Filed: Jun. 20, 2007

Related U.S. Application Data
(63) Continuation of application No. 09/967,305, filed on Sep. 28, 2001, now abandoned.
(60) Provisional application No. 60/236,238, filed on Sep. 28, 2000.

(51) Int. Cl. C12Q 1/68; C12Q 1/00; C12Q 1/02; C07H 21/00; C07K 14/00

(52) U.S. Cl. 435/6; 435/4; 435/29; 536/23.1; 530/350

(57) ABSTRACT

Methods for identifying patients having or at risk of developing prostate cancer (including hormone refractory or androgen independent prostate cancer) and patients having or at risk of developing a cancer arising from metastasis if a prostate cancer to another tissue, e.g., liver and lymph node, by measuring the expression or activity of alpha-methylacyl-CoA racemase are described. The invention also provides: methods of screening for compounds that can be used to treat prostate cancer (including hormone refractory or androgen independent prostate cancer) or metastases of prostate cancer by screening for compounds that modulate the expression or activity of the alpha-methylacyl-CoA racemase polypeptides or nucleic acids; a process for modulating (i.e., reducing) alpha-methylacyl-CoA racemase polypeptide or nucleic acid expression or activity, e.g., using the screened compounds; and methods for selecting patients for therapy with a compound that reduces the activity or expression of alpha-methylacyl-CoA racemase as well as methods for determining whether such a therapy should be continued in a patient.

### CROSS-REFERENCE TO RELATED APPLICATIONS

This application is a continuation of U.S. patent application Ser. No. 09/967,305, filed Sep. 28, 2001, which claims the benefit of U.S. Provisional Application No. 60/236,238, filed on Sep. 28, 2000. The entire contents of each application are incorporated by reference herein.

### TECHNICAL FIELD

This invention relates to detection of prostate cancer, and agents for treating prostate cancer.

### BACKGROUND

Prostate cancer is the most commonly diagnosed cancer and the second most common cause of death from cancer in American men. Prostate cancer cells often initially rely on androgen (e.g., testosterone) for their growth and maintenance. Therefore, androgen withdrawal, by castration or through the use of an anti-androgenic drug, is a common treatment for prostate cancer. In many cases, however, prostate cancer patients develop androgen-independent prostate cancer so that androgen withdrawal treatment is no longer effective. Moreover, metastatic prostate cancer can lead to the formation of tumors in other organs, e.g., liver and lymph nodes.

The complex process of prostate tumor growth and development involves multiple gene products. Therefore, it is important to identify genes involved in tumor development, growth, metastasis and androgen dependence, particularly those genes and gene products that can serve as targets for the diagnosis, prevention, and treatment of prostate cancer.

### SUMMARY

The present invention is based, in part, on the discovery that alpha-methylacyl-CoA racemase (GenBank® Accession No. AF158378 (GI:6653127); AF047020 (GI:4204096)) provides a human alpha-methylacyl-CoA racemase sequence with an alternative amino acid at position 9) is highly expressed in prostate cancer and in metastases of prostate cancer to other tissues (e.g., lung and lymph node). The nucleotide sequence of cDNAs encoding human alpha-methylacyl-CoA racemases are shown in SEQ ID NO:1 (SV1), SEQ ID NO:3 (SV1), and SEQ ID NO:4 (SV1). Amino acid sequence of human alpha-methylacyl-CoA racemase is shown in SEQ ID NO:2 and SEQ ID NO:5. The invention also includes newly discovered splice variants of alpha-methylacyl-CoA racemase (SEQ ID NO:6 (SV2), SEQ ID NO:8 (SV3), and SEQ ID NO:10 (SV4)) and their polypeptide sequences (SEQ ID NO:7 (SV2), SEQ ID NO:9 (SV3); and SEQ ID NO:11 (SV4).

Also included in the invention are nucleic acid sequences and polypeptides that are not shared between various alpha-methylacyl-CoA racemase variants. For example, the amino acid sequence VKASL is unique to SV1 among the alpha-methylacyl-CoA racemase sequences described herein. One method of distinguishing among alpha-methylacyl-CoA racemase polypeptides is by generating an antibody against a fragment unique or partially unique to a particular alpha-methylacyl-CoA racemase. Nucleic acid sequences can also be used to distinguish expression of various alpha-methylacyl-CoA racemase splice variants. For example, SV1, SV2, and SV4 can be distinguished from SV3 with a unique probe from the 486-646 bp region of SV1 which corresponds to the 486-646 bp region of SV2, and corresponds to the 460-622 bp region SV4. Conversely SV3 can be distinguished from SV1, SV2, and SV4 with a probe that includes the splice junction site at 485-486 bp in SV3, e.g., a 20 mer from 475-494 bp of SV3. SV4 can be distinguished from SV1, SV2, and SV3 with a unique probe from the 808-1316 bp region of SV4. SV1 and SV3 can be distinguished from SV2 and SV4 with a unique probe from the 1220-1969 bp region of SV1 which corresponds to the 1059-1808 bp region of SV2. Conversely, SV2 can be distinguished from SV1, SV3, and SV4 with a probe that includes the splice junction site at 1220-1221 in SV2, e.g., a 20mer from 1210-1229 bp of SV2. SV2 can be distinguished from SV1, SV3, and SV4 with a unique probe from the 2400-3654 bp region of SV2. This region is 3′ of the poly A addition signal (at approximately 1290 bp of SV2) which terminates SV1 and is 3′ of the polyA addition signal (at approximately 2400 bp of SV2) which terminates SL3. In general, detection of SV1 is used for applications related to detection of alpha-methylacyl-CoA racemase expression associated with prostate cancer or prostate cancer metastases. Polypeptide sequences encoded by these nucleic acid sequences are useful for, e.g., creating regents such as antibodies that selectively bind to specific splice variants of alpha-methylacyl-CoA racemase.

Alpha-methylacyl-CoA racemase nucleic acid molecules can be used to identify patients having or at risk of developing prostate cancer (including hormone refractory or androgen-independent prostate cancer) and patients having or at risk of developing a cancer arising from metastasis of a prostate cancer (including hormone refractory or androgen-independent prostate cancer) to another tissue, e.g., liver lymph node and bone, as well as other tissues.

In another aspect, the invention provides methods of screening for compounds that can be used to treat prostate cancer (including hormone refractory or androgen-independent prostate cancer) or metastases of prostate cancer by screening for compounds that modulate the expression of the alpha-methylacyl-CoA racemase polypeptides or nucleic acids or the activity of alpha-methylacyl-CoA racemase polypeptides. Compounds that reduce the expression or activity of alpha-methylacyl-CoA racemase are candidate therapeutic compounds. In some cases, additional testing (e.g., in animal models or human patients) can be performed to confirm the ability of the compound to be used to treat prostate cancer. The animal models include rodents (e.g., a mouse or rat) and other non-human mammals with prostate cancer, metastatic prostate cancer (e.g., metastatic prostate cancer to the liver, bone, or lymph node), or a rodent or other non-human mammal harboring a xenograft containing prostate cancer cells, cells from a metastatic prostate cancer, or a prostate cancer cell line. In still another aspect, the invention provides a process for modulating (i.e., reducing) alpha-methylacyl-CoA racemase polypeptide or nucleic acid expression or activity, e.g., using the screened compounds.

The invention also provides assays for determining the activity of or the presence or absence of alpha-methylacyl-CoA racemase polypeptides or nucleic acid molecules in a biological sample, e.g., a sample comprising a cell or a sample comprising polypeptides or nucleic acid molecules (e.g., mRNA) derived or isolated from cells, including for disease diagnosis.

In further aspect the invention provides assays for determining the presence or absence of a genetic alteration in an alpha-methylacyl-CoA racemase polypeptide or nucleic acid molecule, including for disease diagnosis.

The invention also features methods for selecting patients for therapy with a compound that reduces the activity or expression of alpha-methylacyl-CoA racemase and methods for determining whether such a therapy should be continued in a patient.

As used herein, the terms “cancer,” “hyperproliferative,” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is recognized in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. The disorders can arise from poorly differentiated acute leukemias, e.g., erythroblastic leukemia and acute megakaryoblastic leukemia. Exemplary myeloid disorders include, but are not limited to, acute promyeloid leukemia (APML), acute myelogenous leukemia (AML) and chronic myelogenous leukemia (CML) (reviewed in Vaickus (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules that are separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology (John Wiley & Sons, N.Y. (1989), 6.3.1-6.3.6). Aqueous and non-aqueous methods are described in that reference and either can be used. A preferred example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions are hybridization in 6× sodium chloride/sodium citrate (SSC) at about 45° C., followed by one or more washes in 0.2×SSC, 0.1% SDS at 60° C. Preferably, stringent hybridization conditions are hybridization in 6× sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M sodium phosphate, 7% SDS at 65° C., followed by one or more washes at 0.2×SSC, 1% SDS at 65° C. Preferably, an isolated nucleic acid molecule of the invention that hybridizes under stringent conditions to the sequence of SEQ ID NO:1 or SEQ ID NO:3, or the complement thereof corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a alpha-methylacyl-CoA racemase protein, preferably a mammalian alpha-methylacyl-CoA racemase protein, and can further include non-coding regulatory sequences and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of alpha-methylacyl-CoA racemase protein having less than about 30%, 20%, 10%, and more preferably 5% (by dry weight), of non-alpha-methylacyl-CoA racemase protein (also referred to herein as a “contaminating protein”), or of chemical precursors or non-alpha-methylacyl-CoA racemase chemicals. When the alpha-methylacyl-CoA racemase protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of alpha-methylacyl-CoA racemase without abolishing or more preferably, without substantially altering a biological activity, whereas an “essential” amino acid residue results in such a change.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an alpha-methylacyl-CoA racemase protein is preferably replaced with another amino acid residue from the same side chain family. Alternatively, in another embodiment, mutations can be introduced randomly along all or part of an alpha-methylacyl-CoA racemase coding sequence, such as by saturation mutagenesis, and the resultant mutants can be screened for alpha-methylacyl-CoA racemase biological activity to identify mutants that retain activity.

As used herein, a “biologically active portion” of an alpha-methylacyl-CoA racemase protein includes a fragment of a alpha-methylacyl-CoA racemase protein that participates in an interaction between an alpha-methylacyl-CoA racemase molecule and a non-alpha-methylacyl-CoA racemase molecule. Biologically active portions of a alpha-methylacyl-CoA racemase protein include peptides comprising amino acid sequences sufficiently homologous to or derived from the amino acid sequence of the alpha-methylacyl-CoA racemase protein, e.g., the amino acid sequence shown in SEQ ID NO:2, which include fewer amino acids than the full length alpha-methylacyl-CoA racemase proteins, and exhibit at least one activity of a alpha-methylacyl-CoA racemase protein. Typically, biologically active portions comprise a domain or motif with at least one activity of the alpha-methylacyl-CoA racemase protein, e.g., a domain or motif capable of performing a racemization reaction.

A biologically active portion of a alpha-methylacyl-CoA racemase protein can be a polypeptide that is, for example, 10, 25, 50, 100, 200, or more amino acids in length. Biologically active portions of an alpha-methylacyl-CoA racemase protein can be used as targets for developing agents that modulate a alpha-methylacyl-CoA racemase-mediated activity, e.g., a biological activity described herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the alpha-methylacyl-CoA racemase amino acid sequence of SEQ ID NO:2). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available on the internet at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available on the internet at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller (CABIOS (1989) 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to alpha-methylacyl-CoA racemase nucleic acid molecules of the invention. BLAST protein searches can be performed with the XBLAST program, score=50, wordlength=3 to obtain amino acid sequences homologous to alpha-methylacyl-CoA racemase protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. These programs are available on the Internet at www.ncbi.nlm.nih.gov.

“Misexpression or aberrant expression”, as used herein, refers to a non-wild-type pattern of gene expression, at the RNA or protein level (e.g., in a disease tissue such as a prostate tumor or a tumor resulting from metastasis of a prostate tumor). It includes: expression at non-wild-type levels, i.e., over- or under-expression; a pattern of expression that differs from wild-type in terms of the time or stage at which the gene is expressed, e.g., increased or decreased expression (as compared with wild-type) at a predetermined developmental period or stage; a pattern of expression that differs from wild-type in terms of decreased expression (as compared with wild-type) in a predetermined cell type or tissue type; a pattern of expression that differs from wild-type in terms of the splicing size, amino acid sequence, post-translational modification, or biological activity of the expressed polypeptide; a pattern of expression that differs from wild-type in terms of the effect of an environmental stimulus or extracellular stimulus on expression of the gene, e.g., a pattern of increased or decreased expression (as compared with wild-type) in the presence of an increase or decrease in the strength of the stimulus.

An animal, e.g., human, is “at risk” for developing a condition if there is an increased probability that they will develop the condition compared to a population (e.g., the general population, an age-matched population, a population of the same sex). The increased probability can be due to one or a combination of factors including the presence of specific alleles/mutations of a gene or exposure to a particular environment. For example, an individual is at risk for developing androgen independent prostate cancer or androgen independent metastases of prostate cancer when they exhibit increased levels of an alpha-methylacyl-CoA racemase (e.g., SV1) compared to a control population.

The amount of expression of activity of an alpha-methylacyl-CoA racemase in a test cell (e.g., a cell from a prostate tumor) may be evaluated by comparing it to a predetermined value, e.g., the level of expression in a normal prostate cell.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts a cDNA sequence (SEQ ID NO:1) of human alpha-methylacyl-CoA racemase (SV1; GenBank® Accession No. AF158378; GI 6653127). The open reading frame of this sequence extends from nucleotide 66 to nucleotide 1214 (SEQ ID NO:3).

FIG. 2 depicts an amino acid sequence (SEQ ID NO:2) of human alpha-methylacyl-CoA racemase (SV1; GenBank® Accession No. AF158378; GI 6653127).

FIG. 3 depicts a nucleic acid sequence (SEQ ID NO:4) of human alpha-methylacyl-CoA racemase (SV1).

FIG. 4 depicts an amino acid sequence (SEQ ID NO:5) of human alpha-methylacyl-CoA racemase (SV1).

FIGS. 5A-5B depict a nucleic acid sequence (SEQ ID NO:6) of human alpha-methylacyl-CoA racemase (SV2).

FIG. 6 depicts an amino acid sequence (SEQ ID NO:7) of human alpha-methylacyl-CoA racemase (SV2).

FIGS. 7A-7B depict a nucleic acid sequence (SEQ ID NO:8) of human alpha-methylacyl-CoA racemase (SV3).

FIG. 8 depicts an amino acid sequence (SEQ ID NO:9) of human alpha-methylacyl-CoA racemase (SV3).

FIG. 9 depicts a nucleic acid sequence (SEQ ID NO:10) of human alpha-methylacyl-CoA racemase (SV4).

FIG. 10 depicts a predicted amino acid sequence (SEQ ID NO:11) of human alpha-methylacyl-CoA racemase (SV4).

FIG. 11 shows the results of a Western blot of various tissues using antibodies raised against alpha-methylacyl-racemase (ML 185 and ML 186).

FIG. 12 is a schematic drawing comparing SV1, SV2, SV3, and SV4 genomic sequences and illustrating the overlapping sequences between them. Large dark rectangles indicate sequence found in cDNA of the splice variant and small dark rectangles indicate genomic sequence. IMAGE #788180, 1034473, 133130, and 136605 are clones containing alpha-methylacyl-CoA racemase sequence. The light grey rectangles depict the regions in common between the clones and the alpha-methylacyl-CoA racemase sequences illustrated above.

FIG. 13 is an illustration of the predicted organellar targeting features of SV1 and SV2, and the putative transketolase domain of both. S52P and L107P are the locations of human alpha-methylacyl-CoA racemase mutations identified in humans. The location of the peroxisomal targeting signal 1 (PTS1) is indicated and KASL is the single letter amino acid code of the carboxy terminal sequence of SV1 that is the peroxisomal targeting signal.

FIGS. 14 A-D are graphs summarizing microarray analysis of expression patterns of alpha-methylacyl-CoA racemase using sequences from various clones (IMAGE clones 1034473, 788180, 136605 and 133130).

### DETAILED DESCRIPTION

### Example 1

### Expression of Alpha-methylacyl-CoA Racemase in Normal and Cancerous Tissues

The expression of alpha-methylacyl-CoA racemase was measured in various tissue samples using TaqMan® (Applied Biosystems) analysis. These studies analyzed clinical samples taken from prostate cancer patients and from normal individuals. The tissues analyzed included: normal prostate tissue, prostate tumor tissue, normal liver tissue, liver tumor tissue arising from prostate cancer metastasis; normal lymph node tissue, and lymph node tumor tissue arising from prostate cancer metastasis. The TaqMan® reagents used in these experiments detect both SV1 and SV2 mRNAs. As the results in Table 1 and in Table 2 illustrate, alpha-methylacyl-CoA racemase is highly expressed in liver tumors and lymph node tumors arising from prostate cancer metastasis. Such metastases are generally androgen-independent. Alpha-methylacyl-CoA racemase is also more highly expressed in prostate tumors than in normal prostate tissue. Moreover, it appears to be highly expressed in androgen-independent prostate cancer and prostate cancer metastases. Thus, it is highly expressed in prostate cancer that responds poorly (or not at all) to androgen withdrawal therapy.

The expression levels reported for alpha-methylacyl-CoA racemase in Table 1 and in Table 2 are reported as relative expression and are normalized to beta-2 microglobulin expression. The relative expression levels in Table 1 and Table 2 were determined separately and cannot be compared between Tables. Each value reported is for an individual sample.

TABLE 1

| Tissue | Relative Expression |
|---|---|
| Normal Prostate | 0.3, 0.1, 0 |
| Prostate Tumor | 10.5, 9.8, 0.5, 0.7 |
| Normal Liver | 2.6 |
| Liver Tumor (prostate cancer metastasis) | 66.4, 306, 194, 1.9 |
| Normal Lymph Node | 0 |
| Lymph Node Tumor (prostate cancer metastasis) | 24, 0.5, 55.1, 52094 |

TABLE 2

| Tissue | Relative Expression |
|---|---|
| Normal Prostate | 1, 2, 4, 3, 3 |
| Prostate Tumor | 40, 57, 3 |
| Normal Liver | 30, 3 |
| Liver Tumor (prostate cancer metastasis) | 758, 159, 2196, 1532, |
| | 3171, 14 |
| Normal Lymph Node | 16, 1 |
| Lymph Node Tumor (prostate cancer metastasis) | 95, 824, 446, 5, 99, |
| | 451, 20534 |

As shown in Table 3 below, in situ expression analysis of alpha-methylacyl-CoA racemase in clinical samples confirmed TaqMan expression analysis. This analysis also revealed that alpha-methylacyl-CoA racemase is not expressed in normal colon, normal breast, breast tumors, normal lung, and lung tumor. Expression is observed in normal kidney and normal brain tissue. Relatively low level expression was observed in one colon tumor sample.

TABLE 3

| | No. Positive Samples/ |
|---|---|
| Tissue | No. Samples Tested |
| Normal Prostate | 0/3 |
| Prostate Tumor | 6/6 (+/+++) |
| Prostate (liver metastasis) | 5/5 (+++) |
| Prostate (bone metastasis) | 3/4 |
| Normal Colon | 0/3 |
| Colon Tumor | 1/3 (+) |
| Normal Breast | 0/3 |
| Breast Tumor | 0/3 |
| Normal Lung | 0/3 |
| Lung Tumor | 0/1 |
| Normal Brain | 2/2 |
| Normal Liver | 0/2 |
| Normal Kidney | 2/2 |

Expression of alpha-methylacyl-CoA racemase in various tissues was examined using TaqMan technology (Table 5). The tissues examined included aorta, fetal heart, heart (congestive heart failure), vein, aortic smooth muscle cells, spinal cord, brain cortex, glial cells, glioblastoma, breast, breast tumors, ovary, ovary tumor, pancreas, prostate, colon, colon tumor, colon (inflammatory bowel disease), fibrotic liver, fetal liver, lung, lung (chronic obstructive pulmonary disease), spleen, tonsil, lymph node, thymus, epithelial cells, endothelial cells, skeletal muscle, fibroblasts, adipose tissue undifferentiated osteoblasts, and osteoclasts. Expression was particularly high in samples from prostate tumor and brain cortex. Expression was also high in, e.g., kidney, liver, and umbilical vein endothelial cells. In general, the high expression in prostate tumor tissue indicates that alpha-methylacyl-CoA racemase expression may be used to detect prostate tumor.

TABLE 5

| Tissue | Relative Expression |
|---|---|
| Artery (normal) | 1.4 |
| Vein (normal) | 1.2 |
| Aortic smooth muscle cells (early) | 7.8 |
| Coronary smooth muscle cells | 14.8 |
| Static human umbilical vein endothelial cells | 20.3 |
| Shear human umbilical vein endothelial cells | 36.4 |
| Heart (normal) | 1.0 |
| Heart (congestive heart failure) | 4.3 |
| Kidney | 34.4 |
| Skeletal muscle | 13.6 |
| Adipose (normal) | 2.7 |
| Skin normal | 1.1 |
| Osteoclasts (differentiated) | 0.1 |
| Primary osteoblasts | 6.8 |
| Pancreas | 9.2 |
| Spinal cord normal | 2.0 |
| Brain, cortex (normal) | 116.6 |
| Brain, hypothalamus (normal) | 23.1 |
| Nerve | 2.5 |
| DRG (dorsal root ganglion) | 10.2 |
| Glial cells (astrocytes) | 23.4 |
| Glioblastoma | 5.1 |
| Breast (normal) | 4.3 |
| Breast tumor | 3.7 |
| Ovary (normal) | 3.7 |
| Ovary (tumor) | 1.1 |
| Prostate (normal) | 5.4 |
| Prostate (tumor) | 160.4 |
| Epithelial cells (prostate) | 36.5 |
| Colon (normal) | 5.8 |
| Colon (tumor) | 30.7 |
| Lung (normal) | 0.6 |
| Lung (tumor) | 16.4 |
| Lung (chronic obstructive pulmonary disease) | 3.6 |
| Colon (inflammatory bowel disease) | 1.9 |
| Liver (normal) | 30.0 |
| Liver (fibrosis) | 12.8 |
| Dermal cells (fibroblasts) | 2.6 |
| Spleen (normal) | 0.7 |
| Tonsil (normal) | 0.8 |
| Lymph node | 0.5 |
| Resting PBMC | 0.9 |
| Skin-Decubitus | 1.7 |
| Synovium | 0.8 |
| BM-MNC (Bone marrow mononuclear cells) | 0.7 |
| Activated PBMC | 0.1 |

### Example 2

### Immunohistochemical Analysis of Alpha-methylacyl-CoA Racemase

### Immunohistochemical Analysis of Tissue Sections

To further examine expression patterns of alpha-methylacyl-CoA racemase, polyclonal rabbit antibodies (ML185 and ML186) were raised against the enzyme using standard methods. Briefly, the immunogen for generating polyclonal antiserum was a GST fused to the carboxy terminal 38 amino acids of the SV1 form of alpha-methylacyl-CoA racemase. The fusion protein was affinity purified by binding to glutathione Sepharose followed by elution with reduced glutathione. The purified fusion protein was used to immunize rabbits using standard methods.

For use in immunohistochemistry, anti-racemase antibody was affinity purified using a maltose-binding protein-racemase fusion protein immobilized on a column. Anti-GST antibodies were removed by passage over a GST column.

An anti-alpha-methylacyl-CoA racemase antibody was used to immunohistochemically stain and analyze tissue sections from normal prostate, primary prostate tumor, normal tissue in a primary prostate tumor specimen, lymph node metastatic tumor, and lymphocytes adjacent to the lymph node tumor. Briefly, cryostat tissue sections (5 Tm) were fixed in cold acetone (4° C.) for 10 minutes, washed in TBS (0.05 M Tris-HCL, 150 mM NaCl, pH 7.6), and incubated with 3% hydrogen peroxide in methanol to quench endogenous peroxidases, then washed again. Sections were then incubated with blocking buffer (0.5 ml Tween 20, 1.0 g BSA, and 5 ml normal horse serum in 100 ml TBS) followed by incubation with the primary antibody overnight at 4° C. Unbound primary antibody was washed away and biotinylated secondary antibody added, followed by washing and incubation with streptavidin-peroxidase conjugates. The ABC Elite Kit from Vector Laboratories was used as the detection system and the manufacturer's instructions were followed with regard to dilutions and incubation times. The immunoreaction was detected by incubation with substrate (2′,2′-diaminobenzidine) for 10 minutes. Slides were counterstained with hematoxylin and coverslipped with an aqueous mounting medium. The prostate tumor tissue was obtained from prostatectomies and the androgen sensitivity of the tumors was not determined.

Table 4 summarizes the results of these experiments, illustrating that in general, there is greater immunohistochemically detectable alpha-methylacyl-CoA racemase in tumor samples than in prostatic hyperplasia or normal prostate tissue.

These data demonstrate that antibody staining can be a useful method to assist in diagnosis of tumor type and to aid in the determination of an appropriate treatment regimen for an individual that has a tumor expressing alpha-methylacyl-CoA racemase, e.g., an androgen-independent tumor.

TABLE 4

| Category of Tissue | +++ | ++ | + | − |
|---|---|---|---|---|
| Normal prostate/benign prostatic hyperplasia (n = 2) | | | 1 | 1 |
| Normal prostate gland in tumor section (n = 9) | | | 2 | 7 |
| Primary prostate tumor (n = 10) | | 3 | 2 | 5 |
| Lymph node metastases of prostate tumor (n = 7) | 1 | 3 | 1 | 2 |
| Lymphocytes adjacent to lymph node metastasis | | | | 3 |

### Western Blot Analysis

Western blots were performed using the MP 185 and MP 186 antibodies raised against alpha-methylacyl-CoA racemase using standard techniques known to those in the art and as described above. Briefly, Western blots were performed using frozen tissue. Tissue sections were lysed using a Dounce homogenizer in a buffer containing 1% NP40, 50 mM HEPES pH 7.5, 150 mM NaCl, 1 mM EDTA, 5% glycerol, 0.1 mM dithiothreitol (DTT), and protease inhibitors. Lysates were clarified by high speed centrifugation and a portion of the soluble fraction was fractionated by SDS-PAGE and immunoblotted using the indicated antiserum. Samples were not normalized for protein content.

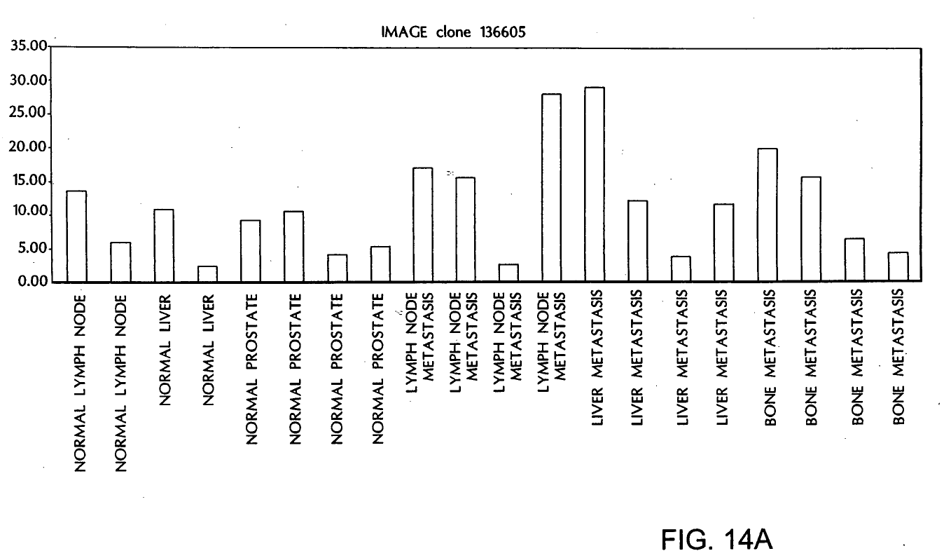

The results of these experiments demonstrate that there is elevated alpha-methylacyl-CoA racemase protein expression in prostate adenocarcinomas and metastases relative to normal prostate tissue (FIG. 11). They also demonstrate that alpha-methylacyl-CoA racemase is expressed in normal kidney and, to a lesser extent, in normal liver. These data also show that Western blot methods can be useful for, e.g., determining treatment regimes by revealing whether a tumor is likely to be sensitive to agents aimed at reducing the expression or activity of alpha-methylacyl-CoA racemase. Such methods can also be used to confirm that an agent can decrease alpha-methylacyl-CoA racemase expression.

### Example 3

### Splice Variants of Alpha-methylacyl-CoA Racemase

Several clones representing three different splicing variants of alpha-methylacyl-CoA racemase have been discovered. The splice variants were identified using sequencing and bioinformatic analyses of cloned sequences, some of which are available in the public domain (IMAGE clones).

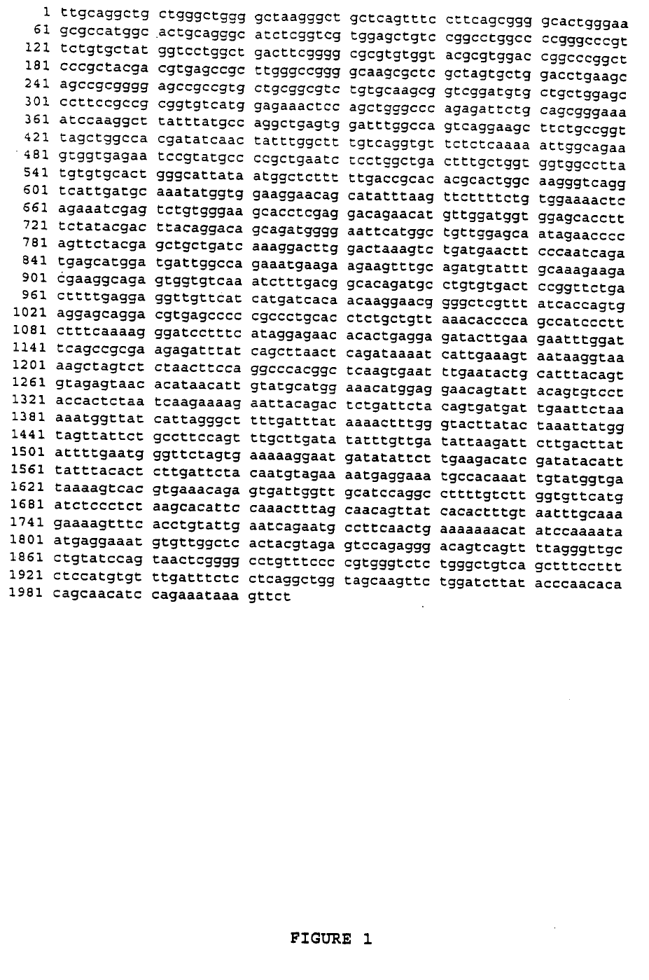

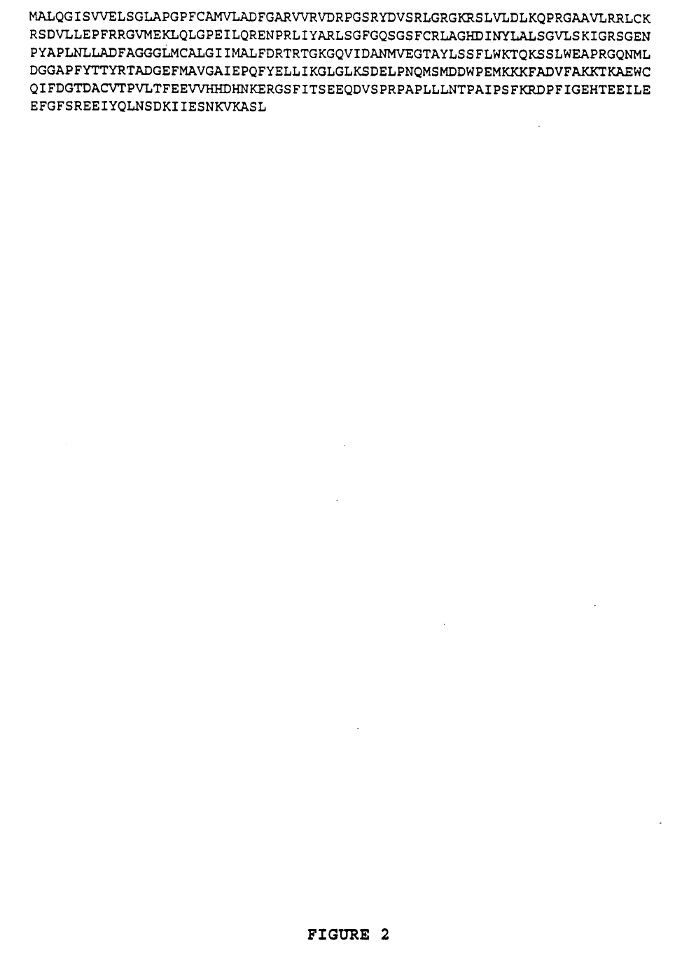

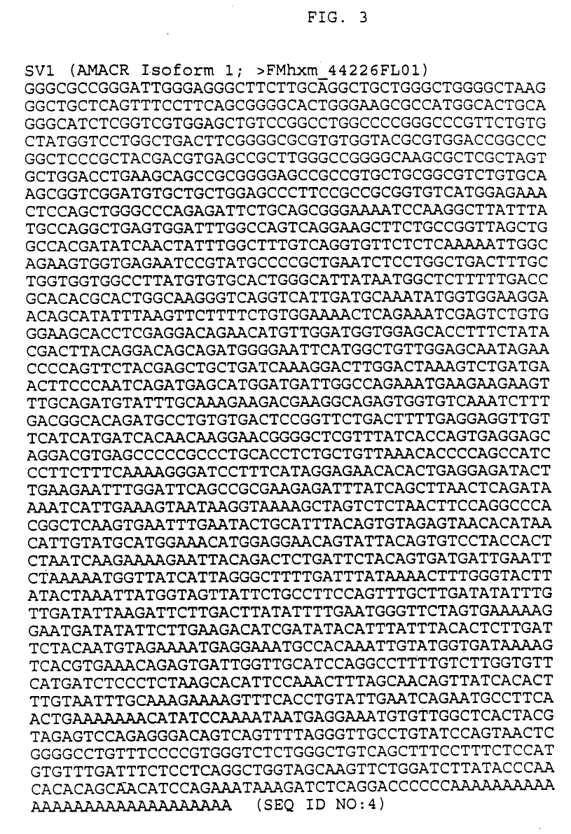

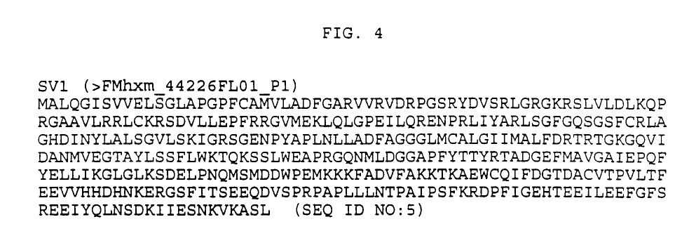

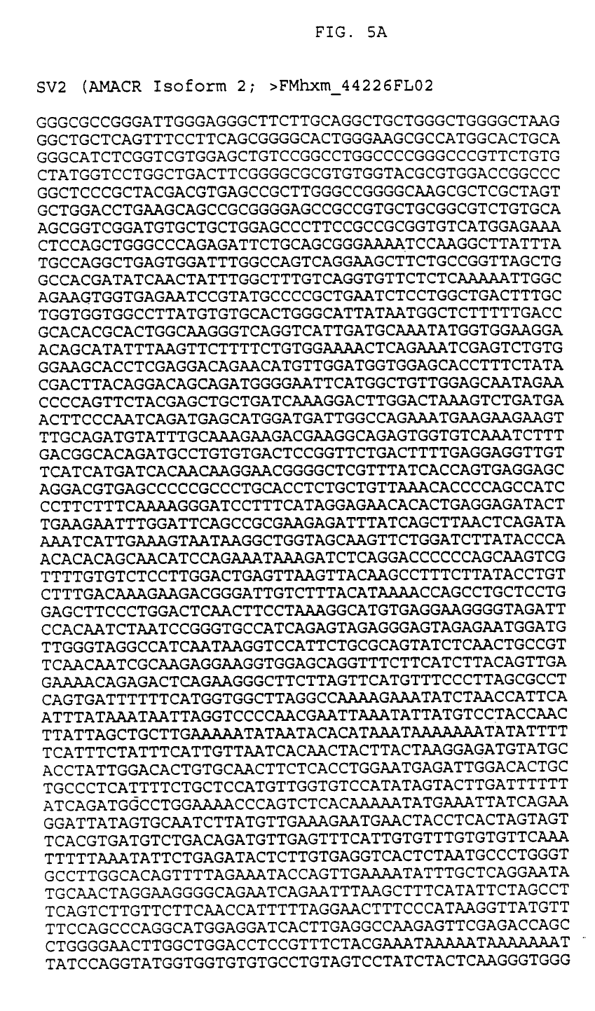

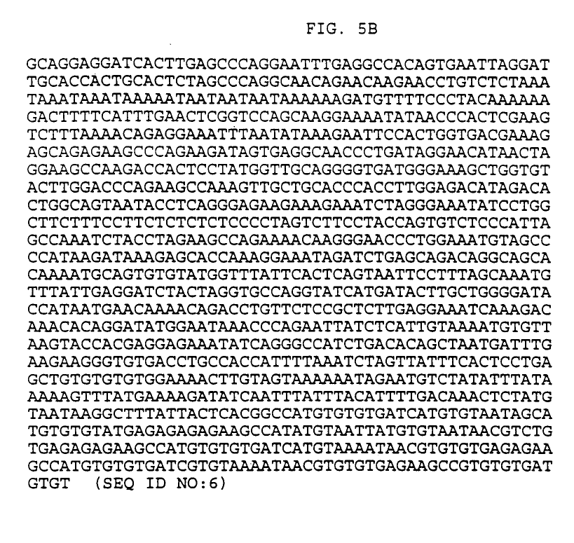

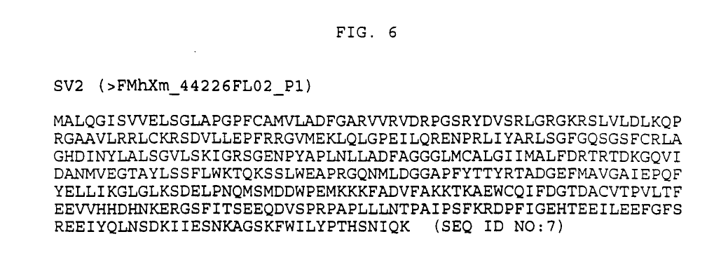

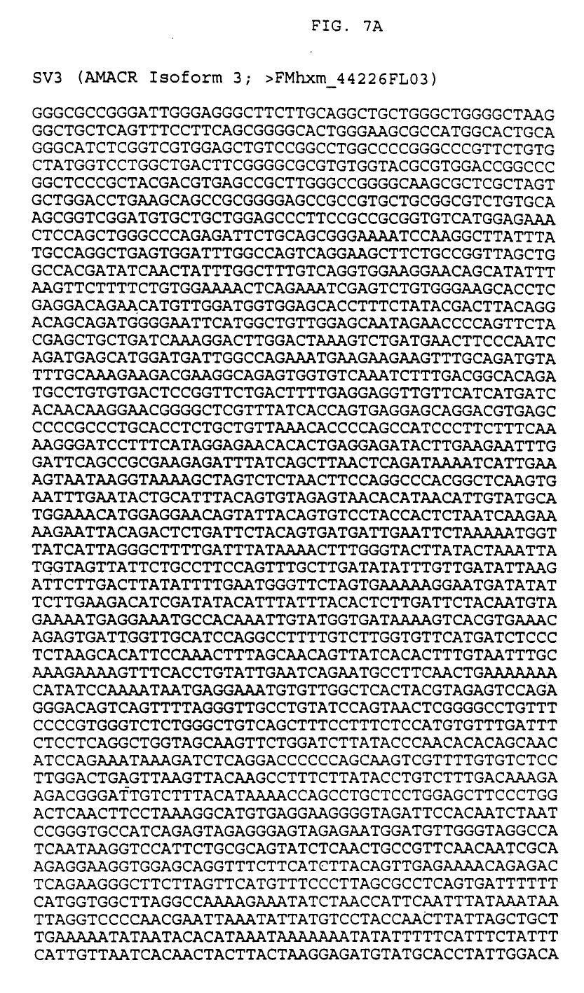

Splice variant 1 (SV1) of alpha-methylacyl-CoA racemase corresponds to published sequence for alpha-methylacyl-CoA racemase (GenBank® Accession Nos. AF158378; GI 6653127). IMAGE clones 788180 and 1034473 contain sequence encoding human alpha-methylacyl-CoA racemase. These sequences can be used to detect expression of alpha-methylacyl-CoA racemase. FIGS. 1 and 2 show the nucleic acid sequence and predicted amino acid sequence, respectively, of SV1. An allele of SV1 is depicted in FIG. 3 (nucleic acid sequence; SEQ ID NO:4) and FIG. 5 (predicted amino acid sequence; SEQ ID NO:6). Based on transcriptional profiling data, this splice variant is elevated in most prostate cancer samples, regardless of clinical stage or PSA (prostate-specific antigen) levels. The sequence is also elevated in tumors resulting from metastases of prostate tumors.

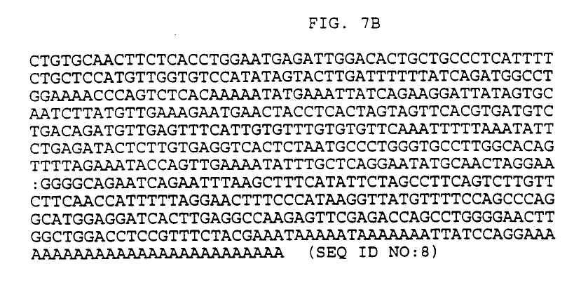

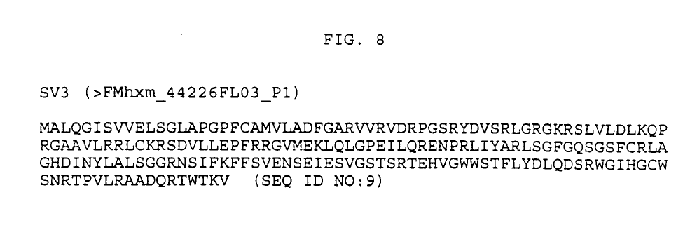

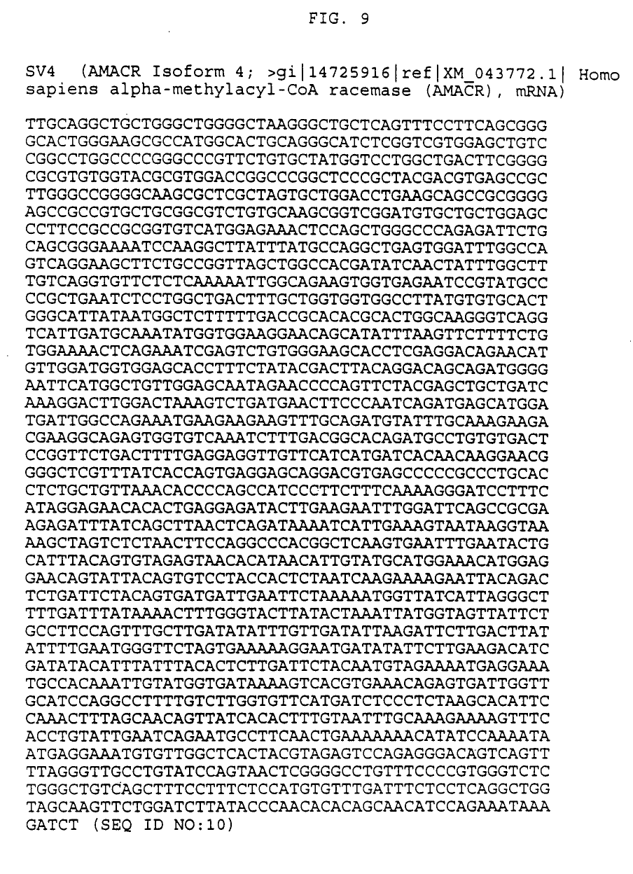

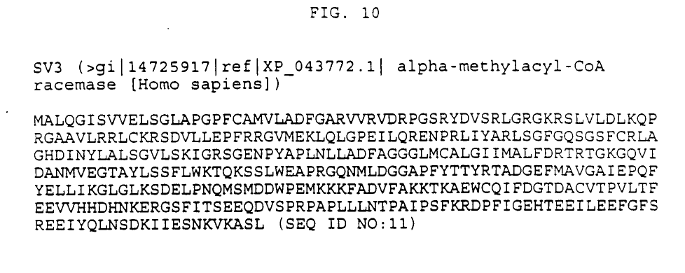

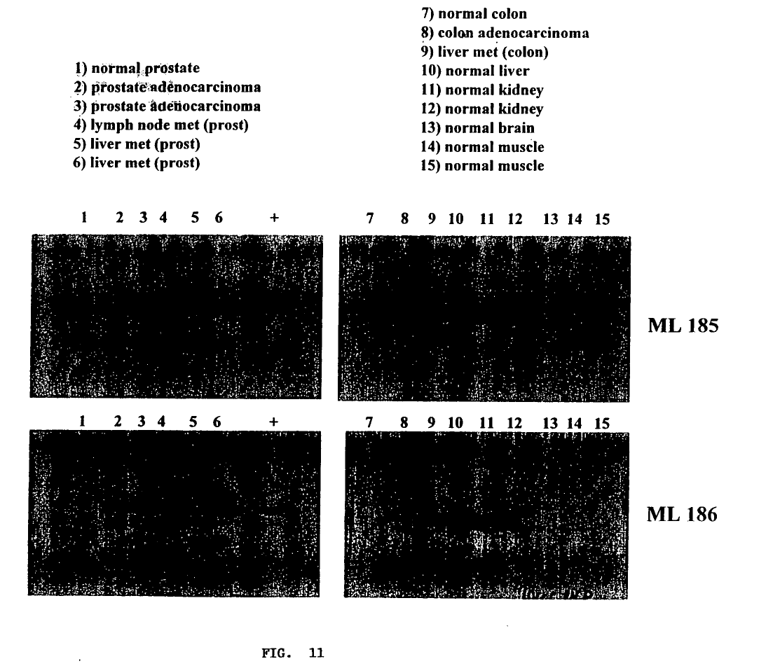

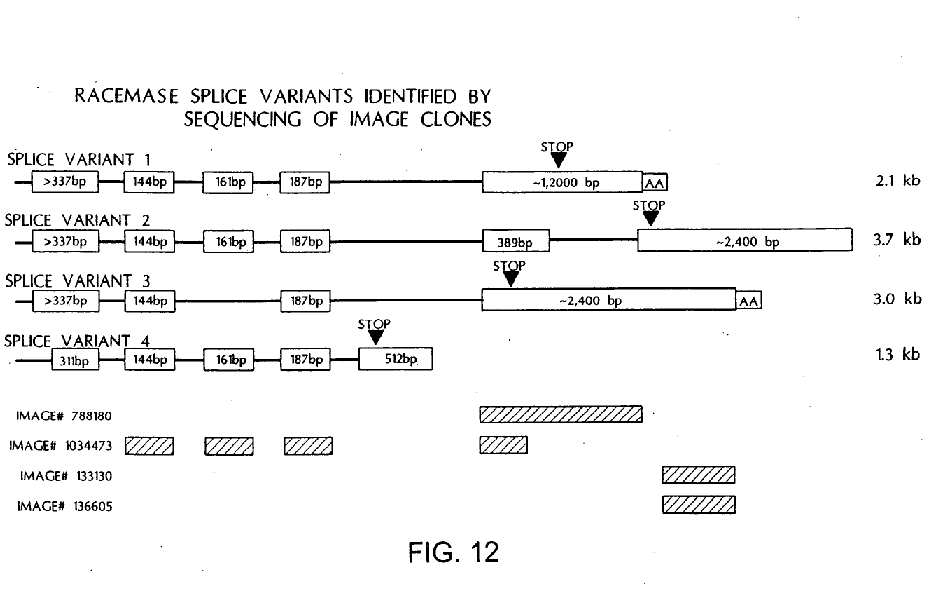

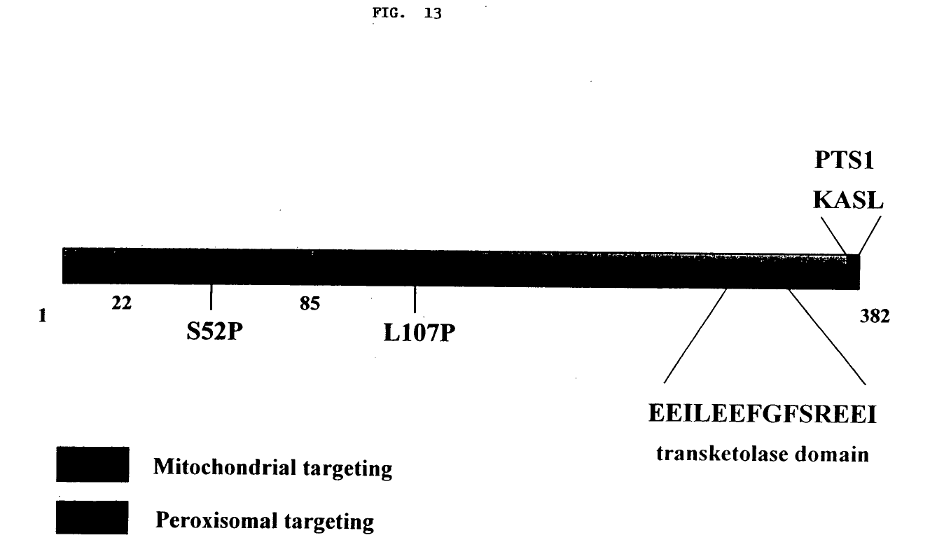

The cDNA sequence of a splice variant of alpha-methylacyl-CoA racemase (SV2) is shown in FIG. 5A-5B (SEQ ID NO:6) and the predicted amino acid sequence is shown in FIG. 6 (SEQ ID NO:7). SV2 is a novel sequence having an additional intron (compared to SV1) that results in a frameshift after amino acid 377, followed by a 17 amino acid extension and a stop codon at position 395. This variant is represented in the arrays used for these analyses by IMAGE clones 133130 and 136605. SV2 shows little or no association with prostate cancer. FIG. B shows a comparison of the predicted translation products for SV1 and SV2. FIG. 5 is a schematic drawing illustrating splice variants identified by sequencing of various SV1 and SV2.

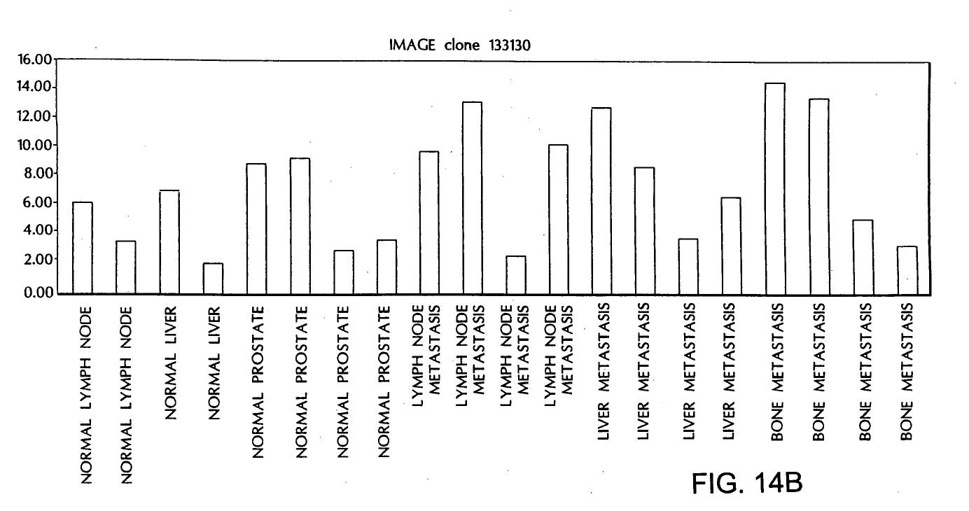

Splice variant 3 is a rare variant that is not predicted to encode a functional alpha-methylacyl-CoA racemase. A fourth sequence has been identified that contains 3′-untranslated region of an alpha-methylacyl-CoA transcript. FIG. 12 shows a schematic comparison of SV1, SV2, and SV3, illustrating the overlapping sequences between them. Such data are useful for, e.g., constructing nucleic acid fragments or antigenic fragments useful for making probes that specifically detect a particular splice variant. The last four amino acids of SV1 are predicted to be peroxisomal targeting signals (Amery et al. (2000) J. Lipid Res. 41:1752). A mitochondrial targeting signal located between amino acids 22 and 85 of alpha-methylacyl-CoA racemase is unmasked when the peroxisomal targeting signal is removed or obscured by the fusion of a GFP protein to the carboxy terminus of SV1. Since the protein product of SV2 lacks the last five amino acids of SV1, including the peroxisomal targeting signal, it is predicted that the SV2 product is mitochondrial in location. FIG. 13 is a drawing illustrating the predicted organellar targeting features of SV1 and SV2. The amino acid sequence is common between SV1 and SV2 except at the carboxy terminus as described herein. SV1 contains the peroxisomal targeting signal (PTS1) which has the sequence KASL. Antibodies targeted to this region may be useful for distinguishing between expression and localization of SV1 and SV2. S52P and L107P are the locations of human alpha-methylacyl-CoA racemase mutations identified in humans (Ferdinandusse et al. (2000) Nature Genet. 24:188). Humans carrying these mutations lack alpha-methylacyl-CoA racemase activity yet survive into adulthood. This shows that agents useful for treating diseases by decreasing the expression or activity of the enzyme are likely to be tolerated by the treated animal (e.g., human).

Microarray analysis was used to analyze the expression patterns of alpha-methylacyl-CoA racemase. cDNA arrays were constructed and alpha-methylacyl-CoA expression was analyzed using standard techniques (e.g., Chiang (2001) Proc. Natl. Acad. Sci. USA 98:2814-2819). The array elements were purified PCR products prepared from plasmid templates. Vector oligonucleotide primers flanking the cloning site in the plasmids were used to amplify the cDNA inserts. Following purification of the PCT product, each template was arrayed onto nylon filters (Biodyne B, Life Technologies, Rockville, Md.) at a density of 6,144 elements per filter. After the filters were dry, the arrayed DNA was denatured in 0.4 M sodium hydroxide, neutralized in 0.1 M Tris.HCl, pH 7.5, rinsed in 2×SSC, and dried to completion. Total RNA was isolated from cultured cells or human tissue specimens. P³³-labeled cDNA was prepared from 2 Tg RNA with SuperScript II (Life Technologies) using both oligo(dT)30 and random primers. After purification over CHROMA SPIN+TE-30 columns (CLONTECH), the labeled cDNA was annealed at 65° C. for 1 hour with 10 μg poly(dA)>200 bases, and 10 μg Cot 10 DNA. 3-6×10⁶ cpm of the annealed cDNA mixture was then added to each array filter. Following overnight hybridization at 65° C., the filters were washed and dried. Dried filters were exposed to phosphoimage screens and the radioactive hybridization signals captured by a Fuji BAS 2500 phosphoimager (Fuji Medical Systems, Stamford, Conn.) and analyzed using software developed at Millennium.

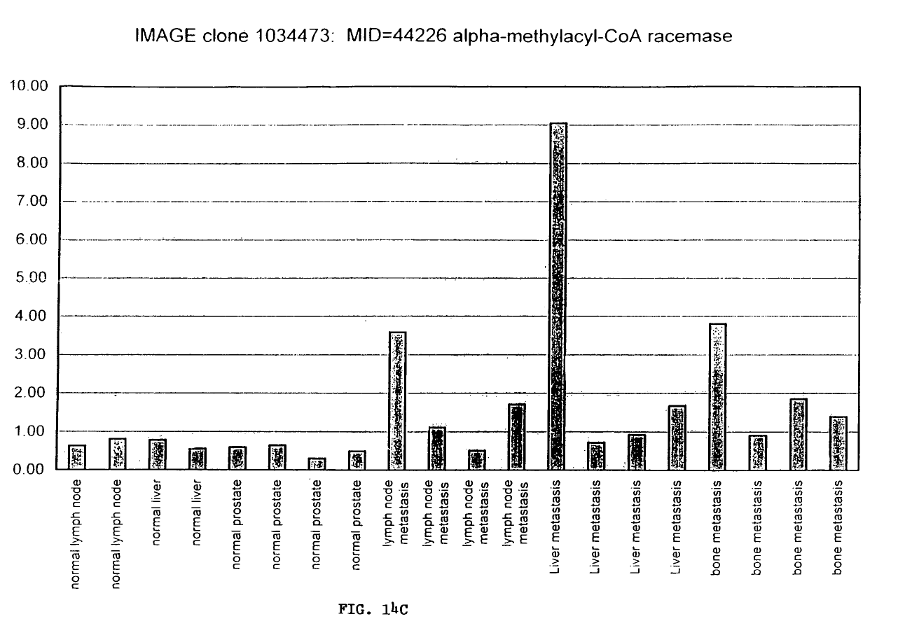

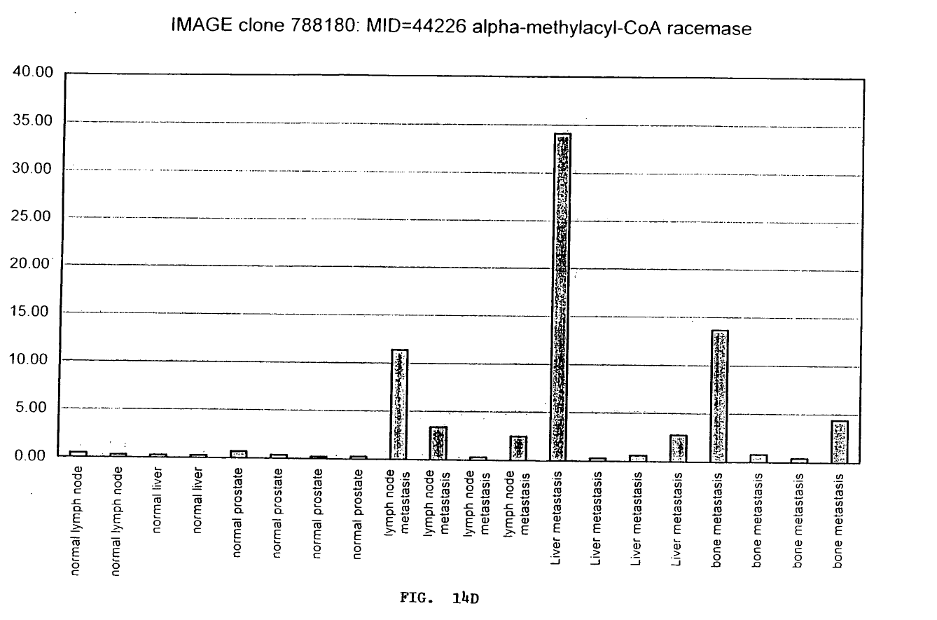

The results of the microarray analysis are show in FIGS. 14A-14D. The tissue samples analyzed were from two normal lymph nodes, two normal livers, four normal prostate glands, four prostate cancer metastases to the lymph node, four prostate cancer metastases to the liver, and four prostate cancer metastases to the bone. The sequences used to detect alpha-methylacyl-CoA racemase expression were from IMAGE clones 136605 (FIG. 14A), 133130 (FIG. 14B), 1034473 (FIG. 14C), and 788180 (FIG. 14D). Clones 133130 and 13605 do not contain SV1 sequence (see FIG. 12) so are not predicted to detect SV1 expression. These data show that alpha-methylacyl-CoA racemase is more highly expressed in a significant number of samples from metastatic prostate cancers.

### Example 4

### Assays for Alpha-methylacyl-CoA Racemase Activity

Alpha-methylacyl-CoA racemase interconverts the (R)- and (S)-isomers of alpha-methyl branched fatty acids when they are in the form of coenzyme A thioesters. One of the roles of alpha-methylacyl-CoA racemase arises as follows. Catabolism of isoprenoids generates branched chain fatty acids, including phytanic acid (3,7,11,15-tetramethylhexadecanoic acid). Phytanic acid undergoes alpha-oxidation to yield pristinic acid (2,6,10,14-tetramethylpentadecanoic acid), which is further degraded by beta-oxidation. However, because pristinic acid is produced as a mixture of (R) and (S) diastereomers and because the oxidases and dehydrogenases responsible for its beta-oxidation act only on the (S)-isomer, a racemase must be available to convert the (R)-isomer. Alpha-methylacyl-CoA racemase serves this function (Schmitz et al. (1995) Eur. J. Biochem. 231:815)

Schmitz et al. (Eur. J. Biochem. 222:313, 1994) describes an assay for alpha-methylacyl-CoA racemase activity. Briefly, (R)- or (S)-2-methyltetradecanoyl-CoA (100 nM) is incubated with alpha-methylacyl-CoA racemase for 1 hour in 200 microliters of 50 mM sodium/potassium phosphate (pH 7.2). The reaction is stopped by the addition of 400 microliters of 6 M HCl followed by heating to 80° C. for 2 hours. The sample is then extracted twice with 0.6 ml of ethyl acetate. The solvent is then evaporated and 0.5 ml of 30 mM carbonyldiimidazole in tolulene is added. Following 10 minutes of incubation at room temperature, the sample is acidified with 10 microliters of glacial acetic acid. Next, 50 microliters of (R)-1-phenylethylamine is added. After a 30 minute incubation at room temperature, the sample is mixed with 5 ml of 50 mM sodium/potassium phosphate (pH 7.5) and then extracted with 1 ml of ethyl acetate, dried under a stream of nitrogen, dissolved in 50 microliters of ethanol, and analyzed by GLC (25 m SE-30 column, 0.32 mm I.D.; isothermal at 240° C.; nitrogen carrier gas at a column head pressure of 80 kPa; direct column injection; flame ionization detector (Veldhoven et al. (1997) Biochem. Biophys. Acta 1347:62; Ferdinandusse et al. (Nature Genetics 24:188, 200).

A useful alternative assay for alpha-methylacyl CoA racemase is the coupled assay described by Veldhoven et al. (1997) Biochem Biophys Acta 1347:62). In this assay alpha-methylacyl racemase is combined with 2R-methyl-pentadecanoyl-CoA. The reaction product, a 2S-isomer, is desaturated by an excess of added oxidase (pristanoyl CoA oxidase) resulting in the production of hydrogen peroxide, which is monitored by means of peroxidase production in the presence of a suitable hydrogen donor. Briefly, alpha-methylacyl CoA racemase is incubated at 30° C. in a reaction mixture containing 0.1 mM 2R-methyl-pentadecanoyl-CoA, 150 mM potassium phosphate (pH 8.0), 25 Tg/ml pristanoyl CoA oxidase, 1 TM FAD, 8 mM tribromohydroxybenzoate.Na salt 2 mM 4-aminoantipyrine, and 40 Tg/ml horseradish peroxidase for a suitable period of time. Formation of the peroxidase reaction product is followed by measuring absorbance at 511 nm.

### Isolated Nucleic Acid Molecules

Various methods of the invention employ an isolated or purified, nucleic acid molecule that encodes an alpha-methylacyl-CoA racemase, e.g., a full-length alpha-methylacyl-CoA racemase protein or a fragment thereof, e.g., a biologically active portion of alpha-methylacyl-CoA racemase protein as well as nucleic acid molecules which hybridize, e.g., under highly stringent conditions, to a nucleic acid molecule that encodes alpha-methylacyl-CoA racemase and nucleic acid molecules having a defined degree of sequence identity (e.g., at least about 60%, 65%, 70%, 75%, 80%, 85%, 90%, 91%, 92%, 93%, 94%, 95%, 96%, 97%, 98%, 99% identity), to a nucleic acid molecule encoding an alpha-methylacyl-CoA racemase (e.g., SEQ ID NO:1; SEQ ID NO:3; SEQ ID NO:4, SEQ ID NO:6, SEQ ID NO:8, and SEQ ID NO:10).

Alpha-methylacyl-CoA racemase probes and primers are useful in many detection methods. Typically a probe/primer is an isolated or purified oligonucleotide. The oligonucleotide typically includes a region of nucleotide sequence that hybridizes under stringent conditions to at least about 7, 12, or 15, preferably about 20 or 25, more preferably about 30, 35, 40, 45, 50, 55, 60, 65, or 75 consecutive nucleotides of a sense or antisense sequence of SEQ ID NO:1, SEQ ID NO:3, SEQ ID NO:4, SEQ ID NO:6, SEQ ID NO:8, or SEQ ID NO:10, or of a naturally occurring allelic variant or mutant of SEQ ID NO:1, SEQ ID NO:3, SEQ ID NO:4, SEQ ID NO:6, SEQ ID NO:8, or SEQ ID NO: 10.

Primers suitable for use in a PCR, which can be used to amplify a selected region of an alpha-methylacyl-CoA racemase sequence, are useful in certain methods of the invention. The primers should be at least 5, 10, or 50 base pairs in length and less than 100, or less than 200, base pairs in length.

Other useful nucleic acid molecules are greater than 260, 300, 400, 500, 600, 700, 800, 900, 1000, or 1100 or more nucleotides in length and hybridize under stringent hybridization conditions to a nucleic acid molecule of SEQ ID NO:1, SEQ ID NO:3, SEQ ID NO:4, SEQ ID NO:6, SEQ ID NO:8, or SEQ ID NO:10.

Nucleic acid molecules comprising or consisting of 100, 200, 300, 400, 500, 600, 700, 800, 900, 1000, or 1100 or more contiguous nucleotides of SEQ ID NO:1, SEQ ID NO:3, SEQ ID NO:4, SEQ ID NO:6, SEQ ID NO:8, or SEQ ID NO:10 are also useful in the methods of the invention.

Also useful in the methods of the invention nucleic acid molecules that differ from the nucleotide sequence shown in SEQ ID NO: 1, SEQ ID NO:3, or SEQ ID NO:4 but still encode the amino acid sequence of SEQ ID NO:2. Other useful nucleic acid molecules encode a protein having an amino acid sequence which differs, by at least 1, but less than 5, 10, 20, 50, or 100 amino acid residues that shown in SEQ ID NO:2, SEQ ID NO:5, SEQ ID NO:7, SEQ ID NO:9, or SEQ ID NO:10. Other useful variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Useful allelic variants of alpha-methylacyl-CoA racemase include both functional and non-functional proteins. Functional allelic variants are naturally occurring amino acid sequence variants of the alpha-methylacyl-CoA racemase protein within a population that maintain the ability to mediate any alpha-methylacyl-CoA racemase biological activity. Functional allelic variants will typically contain only conservative substitution of one or more amino acids of SEQ ID NO:2, SEQ ID NO:5, SEQ ID NO:7, SEQ ID NO:9, or SEQ ID NO:10, or substitution, deletion or insertion of non-critical residues in non-critical regions of the protein. Non-functional allelic variants are naturally-occurring amino acid sequence variants of the alpha-methylacyl-CoA racemase protein within a population that do not have the ability to mediate any alpha-methylacyl-CoA racemase biological activity. Non-functional allelic variants will typically contain a non-conservative substitution, a deletion, or insertion, or premature truncation of the amino acid sequence of SEQ ID NO:2, SEQ ID NO:5, SEQ ID NO:7, SEQ ID NO:9, or SEQ ID NO:10, or a substitution, insertion, or deletion in critical residues or critical regions of the protein.

### Antisense Nucleic Acid Molecules, Ribozymes and Modified Alpha-Methylacyl-CoA Racemase Nucleic Acid Molecules

Isolated nucleic acid molecule that are antisense to alpha-methylacyl-CoA racemase are useful for reducing activity or expression of alpha-methylacyl-CoA racemase. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire alpha-methylacyl-CoA racemase coding strand, or to only a portion thereof (e.g., the coding region of human alpha-methylacyl-CoA racemase). In another embodiment, the antisense nucleic acid molecule is antisense to a “noncoding region” of the coding strand of a nucleotide sequence encoding alpha-methylacyl-CoA racemase (e.g., the 5′ and 3′ untranslated regions).

An antisense nucleic acid can be designed such that it is complementary to the entire coding region of alpha-methylacyl-CoA racemase mRNA, but more preferably is an oligonucleotide that is antisense to only a portion of the coding or noncoding region of alpha-methylacyl-CoA racemase mRNA. For example, the antisense oligonucleotide can be complementary to the region surrounding the translation start site of alpha-methylacyl-CoA racemase mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules are typically administered to a subject (e.g., by direct injection at a tissue site), or generated in situ such that they hybridize with or bind to cellular RNA (e.g., mRNA) and/or genomic DNA encoding an alpha-methylacyl-CoA racemase protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

An antisense nucleic acid can also be a ribozyme. A ribozyme having specificity for a alpha-methylacyl-CoA racemase-encoding nucleic acid can include one or more sequences complementary to the nucleotide sequence of a alpha-methylacyl-CoA racemase cDNA disclosed herein (i.e., SEQ ID NO:1, SEQ ID NO:3, or SEQ ID NO:4), and a sequence having known catalytic sequence responsible for mRNA cleavage (see, for example, U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a alpha-methylacyl-CoA racemase-encoding mRNA (see, e.g., Cech et al. U.S. Pat. No. 4,987,071; and Cech et al. U.S. Pat. No. 5,116,742). Alternatively, alpha-methylacyl-CoA racemase mRNA can be used to select a catalytic RNA having a specific ribonuclease activity from a pool of RNA molecules (see, e.g., Bartel and Szostak (1993) Science 261:1411-1418).

Alpha-methylacyl-CoA racemase gene expression can be inhibited by targeting nucleotide sequences complementary to the regulatory region of the alpha-methylacyl-CoA racemase (e.g., the alpha-methylacyl-CoA racemase promoter and/or enhancers) to form triple helical structures that prevent transcription of the alpha-methylacyl-CoA racemase gene in target cells (see generally, Helene (1991) Anticancer Drug Des. 6(6):569-84; Helene et al. (1992) Ann. N.Y. Acad. Sci. 660:27-36; and Maher (1992) Bioassays 14(12):807-15). The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Detectably labeled oligonucleotide primer and probe molecules are useful in the methods of the invention, e.g., diagnostic methods. Typically, such labels are chemiluminescent, fluorescent, radioactive, or colorimetric.

An alpha-methylacyl-CoA racemase nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4 (1): 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996) supra and Perry-O'Keefe et al. (1996, Proc. Natl. Acad. Sci. 93: 14670-14675).

PNAs of alpha-methylacyl-CoA racemase nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of alpha-methylacyl-CoA racemase nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al. (1996) supra; Perry-O'Keefe supra).

The oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Also useful in the methods of the invention are molecular beacon oligonucleotide primer and probe molecules having at least one region which is complementary to a alpha-methylacyl-CoA racemase nucleic acid of the invention, two complementary regions, one having a fluorophore and one a quencher such that the molecular beacon is useful for quantitating the presence of the alpha-methylacyl-CoA racemase nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

### Isolated Alpha-Methylacyl-CoA Racemase Polypeptides

Isolated alpha-methylacyl-CoA racemase protein, or a fragment, e.g., a biologically active portion thereof, can be used as an immunogen or antigen to raise or test (or more generally to bind) anti-alpha-methylacyl-CoA racemase antibodies useful in diagnostic assays and the preparation of therapeutic compositions. Alpha-methylacyl-CoA racemase protein can be isolated from cells or tissue sources using standard protein purification techniques. Alpha-methylacyl-CoA racemase protein or fragments thereof can be produced by recombinant DNA techniques or synthesized chemically. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present when expressed in a native cell.

Useful alpha-methylacyl-CoA racemase protein or fragments thereof differ from the corresponding sequence in SEQ ID NO:2, SEQ ID NO:5, SEQ ID NO:7, SEQ ID NO:9, or SEQ ID NO:10 (e.g., it differs by at least one, but by less than 15, 10, or 5 amino acid residues or by at least one residue but less than 20%, 15%, 10% or 5% of the residues in it). Useful proteins include an amino acid sequence at least about 60%, 65%, 70%, 75%, 80%, 85%, 90%, 95%, 98%, or more homologous to SEQ ID NO:2, SEQ ID NO:5, SEQ ID NO:7, SEQ ID NO:9, or SEQ ID NO:10.

### Alpha-Methylacyl-CoA Racemase Chimeric or Fusion Proteins

Alpha-methylacyl-CoA racemase chimeric or fusion proteins can also me used in the methods of the invention. As used herein, an alpha-methylacyl-CoA racemase “chimeric protein” or “fusion protein” includes a alpha-methylacyl-CoA racemase polypeptide linked to a non-alpha-methylacyl-CoA racemase polypeptide. A “non-alpha-methylacyl-CoA racemase polypeptide” refers to a polypeptide having an amino acid sequence corresponding to a protein which is not substantially homologous to the alpha-methylacyl-CoA racemase protein, e.g., a protein which is different from the alpha-methylacyl-CoA racemase protein and which is derived from the same or a different organism. The alpha-methylacyl-CoA racemase polypeptide of the fusion protein can correspond to all or a portion e.g., a fragment described herein of an alpha-methylacyl-CoA racemase amino acid sequence. In a preferred embodiment, an alpha-methylacyl-CoA racemase fusion protein includes at least one or more biologically active portions of a alpha-methylacyl-CoA racemase protein. The non-alpha-methylacyl-CoA racemase polypeptide can be fused to the N-terminus or C-terminus of the alpha-methylacyl-CoA racemase polypeptide.

The fusion protein can include a moiety that has a high affinity for a ligand. For example, the fusion protein can be a GST-alpha-methylacyl-CoA racemase fusion protein in which the alpha-methylacyl-CoA racemase sequences are fused to the C-terminus of the GST sequences. Such fusion proteins can facilitate the purification of recombinant alpha-methylacyl-CoA racemase. Alternatively, the fusion protein can be a alpha-methylacyl-CoA racemase protein containing a heterologous signal sequence at its N-terminus. In certain host cells (e.g., mammalian host cells), expression and/or secretion of alpha-methylacyl-CoA racemase can be increased through use of a heterologous signal sequence.

Fusion proteins can include all or a part of a serum protein, e.g., an IgG constant region, or human serum albumin.

The alpha-methylacyl-CoA racemase fusion proteins can be incorporated into pharmaceutical compositions and administered to a subject in vivo. Alpha-methylacyl-CoA racemase fusion proteins can be used to affect the bioavailability of a alpha-methylacyl-CoA racemase substrate. Alpha-methylacyl-CoA racemase fusion proteins may be useful therapeutically for the treatment of disorders caused by, for example, (i) aberrant modification or mutation of a gene encoding a alpha-methylacyl-CoA racemase protein; (ii) mis-regulation of the alpha-methylacyl-CoA racemase gene; and (iii) aberrant post-translational modification of a alpha-methylacyl-CoA racemase protein.

Moreover, the alpha-methylacyl-CoA racemase-fusion proteins of the invention can be used as immunogens to produce anti-alpha-methylacyl-CoA racemase antibodies in a subject, to purify alpha-methylacyl-CoA racemase ligands and in screening assays to identify molecules that inhibit the interaction of alpha-methylacyl-CoA racemase with an alpha-methylacyl-CoA racemase substrate.

Expression vectors are commercially available that already encode a fusion moiety (e.g., a GST polypeptide). An alpha-methylacyl-CoA racemase-encoding nucleic acid can be cloned into such an expression vector such that the fusion moiety is linked in-frame to the alpha-methylacyl-CoA racemase protein.

### Variants of Alpha-Methylacyl-CoA Racemase Proteins

Variants of an alpha-methylacyl-CoA racemase polypeptide, e.g., variants that functions as an agonist (mimetics) or as an antagonist can be useful therapeutically. Variants of the alpha-methylacyl-CoA racemase proteins can be generated by mutagenesis, e.g., discrete point mutation, the insertion or deletion of sequences or the truncation of a alpha-methylacyl-CoA racemase protein. An agonist of the alpha-methylacyl-CoA racemase proteins can retain substantially the same, or a subset, of the biological activities of the naturally occurring form of the protein. An antagonist of an alpha-methylacyl-CoA racemase protein can inhibit one or more of the activities of the naturally occurring form of the protein by, for example, competitively modulating a alpha-methylacyl-CoA racemase-mediated activity of an alpha-methylacyl-CoA racemase protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the alpha-methylacyl-CoA racemase protein.

Variants of a alpha-methylacyl-CoA racemase protein can be identified by screening combinatorial libraries of mutants, e.g., truncation mutants, of a alpha-methylacyl-CoA racemase protein for agonist or antagonist activity.

Libraries of fragments e.g., N-terminal, C-terminal, or internal fragments, of an alpha-methylacyl-CoA racemase protein coding sequence can be used to generate a variegated population of fragments for screening and subsequent selection of variants of an alpha-methylacyl-CoA racemase protein.

Variants in which a cysteine residue is added or deleted or in which a residue that is glycosylated is added or deleted are particularly preferred.

Methods for screening gene products of combinatorial libraries made by point mutations or truncation, and for screening cDNA libraries for gene products having a selected property are known to those skilled in the art. Recursive ensemble mutagenesis (REM), a technique which enhances the frequency of functional mutants in the libraries, can be used in combination with the screening assays to identify alpha-methylacyl-CoA racemase variants (Arkin and Yourvan (1992) Proc. Natl. Acad. Sci. USA 89:7811-7815; Delgrave et al. (1993) Protein Engineering 6(3):327-331).

Cell based assays can be exploited to analyze a variegated alpha-methylacyl-CoA racemase library. For example, a library of expression vectors can be transfected into a cell line, e.g., a cell line, which ordinarily responds to alpha-methylacyl-CoA racemase in a substrate-dependent manner. The transfected cells are then contacted with alpha-methylacyl-CoA racemase and the effect of the expression of the mutant on signaling by the alpha-methylacyl-CoA racemase substrate can be detected, e.g., by measuring changes in cell growth and/or enzymatic activity. Plasmid DNA can then be recovered from the cells that score for inhibition, or alternatively, potentiation of signaling by the alpha-methylacyl-CoA racemase substrate, and the individual clones further characterized.

An alpha-methylacyl-CoA racemase polypeptide having a non-wild-type activity, e.g., an antagonist, agonist, or super agonist of a naturally-occurring alpha-methylacyl-CoA racemase polypeptide, e.g., a naturally-occurring alpha-methylacyl-CoA racemase polypeptide, can be used in certain methods of the invention. These can be created by: altering the sequence of an alpha-methylacyl-CoA racemase polypeptide, e.g., altering the sequence, e.g., by substitution or deletion of one or more residues of a non-conserved region, a domain or residue disclosed herein, and testing the altered polypeptide for the desired activity.

### Anti-Alpha-Methylacyl-CoA Racemase Antibodies

Anti-alpha-methylacyl-CoA racemase antibodies can be used diagnostically and may be useful in therapeutic applications. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

The antibody can be a polyclonal, monoclonal, recombinant, e.g., a chimeric or humanized, fully-human, non-human (e.g., murine), or single chain antibody. In a preferred embodiment, it has effector function and can fix complement. The antibody can be coupled to a toxin or imaging agent.

A full-length alpha-methylacyl-CoA racemase protein or, antigenic peptide fragment of alpha-methylacyl-CoA racemase can be used as an immunogen or can be used to identify anti-alpha-methylacyl-CoA racemase antibodies made with other immunogens, e.g., cells, membrane preparations, and the like. The antigenic peptide of alpha-methylacyl-CoA racemase should include at least 8 amino acid residues of the amino acid sequence shown in SEQ ID NO:2, SEQ ID NO:5, SEQ ID NO:7, SEQ ID NO:9, or SEQ ID NO:10, and encompasses an epitope of alpha-methylacyl-CoA racemase. Preferably, the antigenic peptide includes at least 10 amino acid residues, more preferably at least 15 amino acid residues, even more preferably at least 20 amino acid residues, and most preferably at least 30 amino acid residues.

Preferred epitopes encompassed by the antigenic peptide are regions of alpha-methylacyl-CoA racemase are located on the surface of the protein, e.g., hydrophilic regions, as well as regions with high antigenicity. For example, an Emini surface probability analysis of the human alpha-methylacyl-CoA racemase protein sequence can be used to indicate the regions that have a particularly high probability of being localized to the surface of the alpha-methylacyl-CoA racemase protein and are thus likely to constitute surface residues useful for targeting antibody production.

Chimeric, humanized, but most preferably, completely human antibodies are desirable for applications which include repeated administration, e.g., therapeutic treatment (and some diagnostic applications) of human patients.

The anti-alpha-methylacyl-CoA racemase antibody can be a single chain antibody. A single-chain antibody (scFV) may be engineered (see, for example, Colcher, et al. (1999) Ann N Y Acad Sci 880:263-80; and Reiter (1996) Clin Cancer Res 2:245-52). The single chain antibody can be dimerized or multimerized to generate multivalent antibodies having specificities for different epitopes of the same target alpha-methylacyl-CoA racemase protein.

In a preferred embodiment, the antibody has reduced or no ability to bind an Fc receptor, e.g., it is an isotype, subtype, fragment or other mutant, which does not support binding to an Fc receptor, e.g., it has a mutagenized or deleted Fc receptor binding region.

An anti-alpha-methylacyl-CoA racemase antibody (e.g., monoclonal antibody) can be used to isolate alpha-methylacyl-CoA racemase by standard techniques, such as affinity chromatography or immunoprecipitation. Moreover, an anti-alpha-methylacyl-CoA racemase antibody can be used to detect alpha-methylacyl-CoA racemase protein (e.g., in a cellular lysate or cell supernatant) in order to evaluate the abundance and pattern of expression of the protein. Anti-alpha-methylacyl-CoA racemase antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### Recombinant Expression Vectors, Host Cells and Genetically Engineered Cells

Vectors, preferably expression vectors, containing a nucleic acid encoding alpha-methylacyl-CoA racemase are useful for expressing the protein in vitro and in vivo.

The recombinant expression vectors can be designed for expression of alpha-methylacyl-CoA racemase proteins in prokaryotic or eukaryotic cells, e.g., E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in alpha-methylacyl-CoA racemase activity assays, (e.g., direct assays or competitive assays described in detail below), or to generate antibodies specific for alpha-methylacyl-CoA racemase proteins. To maximize recombinant protein expression in E. coli, the protein is expressed in a host bacterial strain with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The alpha-methylacyl-CoA racemase expression vector can be a yeast expression vector, a vector for expression in insect cells, e.g., a baculovirus expression vector, or a vector suitable for expression in mammalian cells.

When used in mammalian cells, the expression vector's control functions are often provided by viral regulatory elements. For example, commonly used viral promoters are derived from polyoma, Adenovirus 2, cytomegalovirus, and Simian Virus 40 (SV40).

Recombinant mammalian expression vector can be used to direct expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

Other useful recombinant expression vectors are designed to produce antisense RAN. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes, see Weintraub et al., Antisense RNA as a molecular tool for genetic analysis, Reviews: Trends in Genetics, Vol. 1(1) 1986.

Under some circumstances it is desirable to produce a host cell which includes a nucleic acid encoding all or part of an alpha-methylacyl-CoA racemase nucleic acid molecule within a recombinant expression vector or an alpha-methylacyl-CoA racemase nucleic acid molecule containing sequences which allow it to homologously recombine into a specific site of the host cell's genome. A host cell can be any prokaryotic or eukaryotic cell. For example, an alpha-methylacyl-CoA racemase protein can be expressed in bacterial cells such as E. coli, insect cells, yeast, or mammalian cells (such as Chinese hamster ovary cells (CHO)) or COS cells. Other suitable host cells are known to those skilled in the art.

Vector DNA can be introduced into host cells via conventional transformation or transfection techniques, e.g., any art-recognized technique for introducing foreign nucleic acid (e.g., DNA) into a host cell, including calcium phosphate or calcium chloride co-precipitation, DEAE-dextran-mediated transfection, lipofection, or electroporation.

The host cell of the invention can be used to produce (i.e., express) an alpha-methylacyl-CoA racemase protein, e.g., by culturing a host cell (into which a recombinant expression vector encoding an alpha-methylacyl-CoA racemase protein has been introduced) in a suitable medium such that an alpha-methylacyl-CoA racemase protein is produced and, optionally isolating a alpha-methylacyl-CoA racemase protein from the medium or the host cell.

A cell or purified preparation of cells which include an alpha-methylacyl-CoA racemase transgene, or which otherwise mis-express alpha-methylacyl-CoA racemase can be used as a model for studying disorders (e.g., prligerative disorders) that are related to mutated or mis-expressed alpha-methylacyl-CoA racemase alleles or for use in drug screening. The cell preparation can consist of human or non-human cells, e.g., rodent cells, such as mouse or rat cells; rabbit cells; or pig cells. In preferred embodiments, the cell or cells include an alpha-methylacyl-CoA racemase transgene, e.g., a heterologous form of a alpha-methylacyl-CoA racemase, e.g., a gene derived from humans (in the case of a non-human cell). The alpha-methylacyl-CoA racemase transgene can be mis-expressed, e.g., overexpressed or underexpressed. The cell or cells can include a gene that mis-expresses an endogenous alpha-methylacyl-CoA racemase, e.g., a gene the expression of which is disrupted, e.g., a knockout.

The expression characteristics of an endogenous gene within a cell, e.g., a cell line or microorganism, can be modified by inserting a heterologous DNA regulatory element into the genome of the cell such that the inserted regulatory element is operably linked to the endogenous alpha-methylacyl-CoA racemase gene. For example, an endogenous alpha-methylacyl-CoA racemase gene that is “transcriptionally silent,” e.g., not normally expressed, or expressed only at very low levels, may be activated by inserting a regulatory element that is capable of promoting the expression of a normally expressed gene product in that cell. Techniques such as targeted homologous recombination, can be used to insert the heterologous DNA as described in, e.g., Chappel, U.S. Pat. No. 5,272,071; WO 91/06667, published in May 16, 1991.

### Transgenic Animals

Non-human transgenic animals expressing human alpha-methylacyl-CoA racemase are useful for studying the function and/or activity of a alpha-methylacyl-CoA racemase protein and for identifying and/or evaluating modulators of alpha-methylacyl-CoA racemase activity. A transgenic animal is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous alpha-methylacyl-CoA racemase gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of an alpha-methylacyl-CoA racemase protein to particular cells. A transgenic founder animal can be identified based upon the presence of a alpha-methylacyl-CoA racemase transgene in its genome and/or expression of alpha-methylacyl-CoA racemase mRNA in tissues or cells of the animals. A transgenic founder animal can then be used to breed additional animals carrying the transgene. Moreover, transgenic animals carrying a transgene encoding a alpha-methylacyl-CoA racemase protein can further be bred to other transgenic animals carrying other transgenes.

Alpha-methylacyl-CoA racemase proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk- or egg-specific promoter, and recovered from the milk or eggs produced by the animal. Suitable animals are mice, pigs, cows, goats, and sheep.

The invention also includes a population of cells from a transgenic animal, as discussed, e.g., below.

### Uses

Alpha-methylacyl-CoA racemase nucleic acid molecules, proteins, protein homologues, and antibodies can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). The isolated alpha-methylacyl-CoA racemase nucleic acid molecules can be used, for example, to express a alpha-methylacyl-CoA racemase protein (e.g., via a recombinant expression vector in a host cell in gene therapy applications), to detect a alpha-methylacyl-CoA racemase mRNA (e.g., in a biological sample), to detect a genetic alteration in an alpha-methylacyl-CoA racemase gene and to modulate alpha-methylacyl-CoA racemase activity, as described further below. The alpha-methylacyl-CoA racemase proteins can be used to treat disorders characterized by excessive production of an alpha-methylacyl-CoA racemase substrate or production of alpha-methylacyl-CoA racemase inhibitors. In addition, the alpha-methylacyl-CoA racemase proteins can be used to screen for naturally occurring alpha-methylacyl-CoA racemase substrates or inhibitors, to screen for drugs or compounds which modulate alpha-methylacyl-CoA racemase activity, as well as to treat disorders characterized by excessive production of alpha-methylacyl-CoA racemase protein or production of alpha-methylacyl-CoA racemase protein forms which have decreased, aberrant or unwanted activity compared to alpha-methylacyl-CoA racemase wild-type protein. Moreover, anti-alpha-methylacyl-CoA racemase antibodies can be used to detect alpha-methylacyl-CoA racemase proteins, regulate the bioavailability of alpha-methylacyl-CoA racemase proteins, and modulate alpha-methylacyl-CoA racemase activity.

A method of evaluating a compound for the ability to interact with, e.g., bind to, a subject alpha-methylacyl-CoA racemase polypeptide is provided. Such compounds can be useful for diagnosis or treatment of a proliferative disorder. The method includes: contacting the compound with alpha-methylacyl-CoA racemase polypeptide; and evaluating the ability of the compound to interact with, e.g., to bind or form a complex with, alpha-methylacyl-CoA racemase polypeptide. This method can be performed in vitro, e.g., in a cell-free system, or in vivo, e.g., in a two-hybrid interaction trap assay. This method can be used to identify naturally-occurring molecules that interact with a subject alpha-methylacyl-CoA racemase polypeptide. It can also be used to find natural or synthetic inhibitors of a subject alpha-methylacyl-CoA racemase polypeptide. Screening methods are discussed in more detail below.

### Screening Assays:

The invention provides screening methods (also referred to herein as “assays”) for identifying modulators, i.e., candidate or test compounds or agents (e.g., proteins, peptides, peptidomimetics, peptoids, small molecules or other drugs) which bind to alpha-methylacyl-CoA racemase proteins, have an inhibitory (or stimulatory) effect on, for example, alpha-methylacyl-CoA racemase expression or alpha-methylacyl-CoA racemase activity, or have a stimulatory or inhibitory effect on, for example, the expression or activity of a alpha-methylacyl-CoA racemase substrate. Compounds thus identified can be used to modulate the activity of target gene products (e.g., alpha-methylacyl-CoA racemase genes) either directly or indirectly in a therapeutic protocol, to elaborate the biological function of the target gene product, or to identify compounds that disrupt normal target gene interactions. Compounds which inhibit the activity or expression of alpha-methylacyl-CoA racemase are useful in the treatment of proliferative disorders, e.g., cancer, particularly metastatic (e.g., androgen-independent) prostate cancer.

In one embodiment, the invention provides assays for screening candidate or test compounds that are substrates of an alpha-methylacyl-CoA racemase protein or polypeptide or a biologically active portion thereof. In another embodiment, the invention provides assays for screening candidate or test compounds that bind to or modulate the activity of a alpha-methylacyl-CoA racemase protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone, which are resistant to enzymatic degradation but which nevertheless remain bioactive; see, e.g., Zuckermann et al. (1994) J. Med. Chem. 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994)Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner, U.S. Pat. No. 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; and Ladner supra.).

In one embodiment, an assay is a cell-based assay in which a cell that expresses an alpha-methylacyl-CoA racemase protein or biologically active portion thereof is contacted with a test compound, and the ability of the test compound to modulate alpha-methylacyl-CoA racemase activity is determined. Determining the ability of the test compound to modulate alpha-methylacyl-CoA racemase activity can be accomplished by monitoring, for example, changes in enzymatic activity. The cell, for example, can be of mammalian origin. The ability of the test compound to modulate alpha-methylacyl-CoA racemase binding to a compound, e.g., an alpha-methylacyl-CoA racemase substrate, or to bind to alpha-methylacyl-CoA racemase can also be evaluated. This can be accomplished, for example, by coupling the compound, e.g., the substrate, with a radioisotope or enzymatic label such that binding of the compound, e.g., the substrate, to alpha-methylacyl-CoA racemase can be determined by detecting the labeled compound, e.g., substrate, in a complex. Alternatively, alpha-methylacyl-CoA racemase could be coupled with a radioisotope or enzymatic label to monitor the ability of a test compound to modulate alpha-methylacyl-CoA racemase binding to an alpha-methylacyl-CoA racemase substrate in a complex. For example, compounds (e.g., alpha-methylacyl-CoA racemase substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., an alpha-methylacyl-CoA racemase substrate) to interact with alpha-methylacyl-CoA racemase with or without the labeling of any of the interactants can be evaluated. For example, a microphysiometer can be used to detect the interaction of a compound with alpha-methylacyl-CoA racemase without the labeling of either the compound or the alpha-methylacyl-CoA racemase (McConnell et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and alpha-methylacyl-CoA racemase.

In yet another embodiment, a cell-free assay is provided in which a alpha-methylacyl-CoA racemase protein or biologically active portion thereof is contacted with a test compound and the ability of the test compound to bind to the alpha-methylacyl-CoA racemase protein or biologically active portion thereof is evaluated. Preferred biologically active portions of the alpha-methylacyl-CoA racemase proteins to be used in assays of the present invention include fragments that participate in interactions with non-alpha-methylacyl-CoA racemase molecules, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label is selected such that a first donor molecule's emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the alpha-methylacyl-CoA racemase protein to bind to a target molecule can be accomplished using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize alpha-methylacyl-CoA racemase, an anti-alpha-methylacyl-CoA racemase antibody or its target molecule to facilitate separation of complexed from non-complexed forms of one or both of the proteins, as well as to accommodate automation of the assay. Binding of a test compound to an alpha-methylacyl-CoA racemase protein, or interaction of an alpha-methylacyl-CoA racemase protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/alpha-methylacyl-CoA racemase fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or alpha-methylacyl-CoA racemase protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of alpha-methylacyl-CoA racemase binding or activity determined using standard techniques.

Other techniques for immobilizing either alpha-methylacyl-CoA racemase protein or a target molecule on matrices include using conjugation of biotin and streptavidin. Biotinylated alpha-methylacyl-CoA racemase protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

This assay is performed utilizing antibodies reactive with alpha-methylacyl-CoA racemase protein or target molecules but which do not interfere with binding of the alpha-methylacyl-CoA racemase protein to its target molecule. Such antibodies can be derivatized to the wells of the plate, and unbound target or alpha-methylacyl-CoA racemase protein trapped in the wells by antibody conjugation. Methods for detecting such complexes, in addition to those described above for the GST-immobilized complexes, include immunodetection of complexes using antibodies reactive with the alpha-methylacyl-CoA racemase protein or target molecule, as well as enzyme-linked assays which rely on detecting an enzymatic activity associated with the alpha-methylacyl-CoA racemase protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (see, e.g., Heegaard (1998) J Mol Recognit 11:141-8; Hage and Tweed (1997) J Chromatogr B Biomed Sci Appl 699:499-525). Further, fluorescence energy transfer may also be conveniently utilized, as described herein, to detect binding without further purification of the complex from solution.

The assay can include contacting the alpha-methylacyl-CoA racemase protein or biologically active portion thereof with a known compound that binds alpha-methylacyl-CoA racemase to form an assay mixture, contacting the assay mixture with a test compound, and determining the ability of the test compound to interact with an alpha-methylacyl-CoA racemase protein, wherein determining the ability of the test compound to interact with an alpha-methylacyl-CoA racemase protein includes determining the ability of the test compound to preferentially bind to alpha-methylacyl-CoA racemase or biologically active portion thereof, or to modulate the activity of a target molecule, as compared to the known compound.

To the extent that alpha-methylacyl-CoA racemase can, in vivo, interact with one or more cellular or extracellular macromolecules, such as proteins, inhibitors of such an interaction are useful. A homogeneous assay can be used can be used to identify inhibitors. For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, alpha-methylacyl-CoA racemase protein can be used as a “bait protein” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, that bind to or interact with alpha-methylacyl-CoA racemase (“alpha-methylacyl-CoA racemase-binding proteins” or “alpha-methylacyl-CoA racemase-bp”) and are involved in alpha-methylacyl-CoA racemase activity. Such alpha-methylacyl-CoA racemase-bps can be activators or inhibitors of signals by the alpha-methylacyl-CoA racemase proteins or alpha-methylacyl-CoA racemase targets as, for example, downstream elements of a alpha-methylacyl-CoA racemase-mediated signaling pathway.

Modulators of alpha-methylacyl-CoA racemase expression can also be identified. For example, a cell or cell free mixture is contacted with a candidate compound and the expression of alpha-methylacyl-CoA racemase mRNA or protein evaluated relative to the level of expression of alpha-methylacyl-CoA racemase mRNA or protein in the absence of the candidate compound. When expression of alpha-methylacyl-CoA racemase mRNA or protein is greater in the presence of the candidate compound than in its absence, the candidate compound is identified as a stimulator of alpha-methylacyl-CoA racemase mRNA or protein expression. Alternatively, when expression of alpha-methylacyl-CoA racemase mRNA or protein is less (i.e., statistically significantly less) in the presence of the candidate compound than in its absence, the candidate compound is identified as an inhibitor of alpha-methylacyl-CoA racemase mRNA or protein expression. The level of alpha-methylacyl-CoA racemase mRNA or protein expression can be determined by methods described herein for detecting alpha-methylacyl-CoA racemase mRNA or protein.

A modulating agent can be identified using a cell-based or a cell free assay, and the ability of the agent to modulate the activity of a alpha-methylacyl-CoA racemase protein can be confirmed in vivo, e.g., in an animal such as an animal model for a disease (e.g., an animal with prostate cancer or metastatic prostate cancer; or an animal harboring a xenograft of a prostate cancer from an animal (e.g., human) or cells from a cancer resulting from metastasis of a prostate cancer (e.g, to a lymph node, bone, or liver), or cells from a prostate cancer cell line.

This invention further pertains to novel agents identified by the above-described screening assays. Accordingly, it is within the scope of this invention to further use an agent identified as described herein (e.g., a alpha-methylacyl-CoA racemase modulating agent, an antisense alpha-methylacyl-CoA racemase nucleic acid molecule, a alpha-methylacyl-CoA racemase-specific antibody, or a alpha-methylacyl-CoA racemase-binding partner) in an appropriate animal model (such as those described above) to determine the efficacy, toxicity, side effects, or mechanism of action, of treatment with such an agent. Furthermore, novel agents identified by the above-described screening assays can be used for treatments as described herein.

### Predictive Medicine

The present invention also pertains to the field of predictive medicine in which diagnostic assays, prognostic assays, and monitoring clinical trials are used for prognostic (predictive) purposes to thereby treat an individual.

Generally, the invention provides a method of determining if a subject is at risk for a disorder related to a lesion in, or the misexpression of, a gene that encodes an alpha-methylacyl-CoA racemase polypeptide.

Such disorders include, e.g., a disorder associated with the misexpression of a alpha-methylacyl-CoA racemase polypeptide, e.g., a neoplastic disorder.

The method includes one or more of the following:

detecting, in a tissue of the subject, the presence or absence of a mutation which affects the expression of the alpha-methylacyl-CoA racemase gene, or detecting the presence or absence of a mutation in a region which controls the expression of the gene, e.g., a mutation in the 5′ control region;

detecting, in a tissue of the subject, the presence or absence of a mutation which alters the structure of the alpha-methylacyl-CoA racemase gene;

detecting, in a tissue of the subject, the misexpression of the alpha-methylacyl-CoA racemase gene at the mRNA level, e.g., detecting a non-wild-type level of a mRNA;

detecting, in a tissue of the subject, the misexpression of the gene at the protein level, e.g., detecting a non-wild-type level of a alpha-methylacyl-CoA racemase polypeptide.

In preferred embodiments the method includes: ascertaining the existence of at least one of: a deletion of one or more nucleotides from the alpha-methylacyl-CoA racemase gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO: 1, SEQ ID NO:4, SEQ ID NO:6, SEQ ID NO:8, or SEQ ID NO:10, or naturally occurring mutants thereof, or 5′ or 3′ flanking sequences naturally associated with the alpha-methylacyl-CoA racemase gene; (ii) exposing the probe/primer to nucleic acid of the tissue; and detecting the presence or absence of the genetic lesion by hybridization of the probe/primer to the nucleic acid, e.g., by in situ hybridization.

In preferred embodiments, detecting the misexpression includes ascertaining the existence of at least one of: an alteration in the level of a messenger RNA transcript of the alpha-methylacyl-CoA racemase gene; the presence of a non-wild-type splicing pattern of a messenger RNA transcript of the gene; or a non-wild-type level of alpha-methylacyl-CoA racemase RNA or protein.

In preferred embodiments the method includes determining the structure of a alpha-methylacyl-CoA racemase gene, an abnormal structure being indicative of risk for the disorder.

In preferred embodiments the method includes contacting a sample form the subject with an antibody to the alpha-methylacyl-CoA racemase protein or a nucleic acid, which hybridizes specifically with the gene. These and other embodiments are discussed below.

### Diagnostic and Prognostic Assays

The presence, level, or absence of alpha-methylacyl-CoA racemase protein or nucleic acid in a biological sample can be evaluated by obtaining a biological sample from a test subject and contacting the biological sample with a compound or an agent capable of detecting alpha-methylacyl-CoA racemase protein or nucleic acid (e.g., mRNA, genomic DNA) that encodes alpha-methylacyl-CoA racemase protein such that the presence of alpha-methylacyl-CoA racemase protein or nucleic acid is detected in the biological sample. The term “biological sample” includes tissues, cells and biological fluids isolated from a subject, as well as tissues, cells and fluids present within a subject. A preferred biological sample is serum. The level of expression of the alpha-methylacyl-CoA racemase gene can be measured in a number of ways, including, but not limited to: measuring the mRNA encoded by the alpha-methylacyl-CoA racemase genes; measuring the amount of protein encoded by the alpha-methylacyl-CoA racemase genes; or measuring the activity of the protein encoded by the alpha-methylacyl-CoA racemase genes. Such methods can measure, e.g., the absolute level or relative level of a nucleic acid, protein, or activity

The level of mRNA corresponding to the alpha-methylacyl-CoA racemase gene in a cell can be determined both by in situ and by in vitro formats.

The isolated mRNA can be used in hybridization or amplification assays that include, but are not limited to, Southern or Northern analyses, polymerase chain reaction analyses, and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length alpha-methylacyl-CoA racemase nucleic acid, such as the nucleic acid of SEQ ID NO:1, SEQ ID NO:3, SEQ ID NO:4, SEQ ID NO:6, SEQ ID NO:8, SEQ ID NO:10 or a portion thereof, such as an oligonucleotide of at least 15, 30, 50, 100, 250, or 500 nucleotides in length and sufficient to specifically hybridize under stringent conditions to alpha-methylacyl-CoA racemase mRNA or genomic DNA. Other suitable probes for use in the diagnostic assays are described herein.

In one format, mRNA (or cDNA) is immobilized on a surface and contacted with the probes, for example, by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the alpha-methylacyl-CoA racemase genes.

The level of mRNA in a sample that is encoded by alpha-methylacyl-CoA racemase can be evaluated with nucleic acid amplification, e.g., by RT-PCR (Mullis, 1987, U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of an alpha-methylacyl-CoA racemase gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence between the primers.

For in situ methods, a cell or tissue sample can be prepared/processed and immobilized on a support, typically a glass slide, and then contacted with a probe that can hybridize to mRNA that encodes the alpha-methylacyl-CoA racemase gene being analyzed.

In another embodiment, the methods include further contacting a control sample with a compound or agent capable of detecting alpha-methylacyl-CoA racemase mRNA, or genomic DNA, and comparing the presence of alpha-methylacyl-CoA racemase mRNA or genomic DNA in the control sample with the presence of alpha-methylacyl-CoA racemase mRNA or genomic DNA in the test sample.

A variety of methods can be used to determine the level of protein encoded by alpha-methylacyl-CoA racemase. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect alpha-methylacyl-CoA racemase protein in a biological sample in vitro as well as in vivo. In vitro techniques for detection of alpha-methylacyl-CoA racemase protein include enzyme linked immunosorbent assays (ELISAs), immunoprecipitations, immunofluorescence, enzyme immunoassay (EIA), radioimmunoassay (RIA), and Western blot analysis. In vivo techniques for detection of alpha-methylacyl-CoA racemase protein include introducing into a subject a labeled anti-alpha-methylacyl-CoA racemase antibody. For example, the antibody can be labeled with a radioactive marker whose presence and location in a subject can be detected by standard imaging techniques.

In another embodiment, the methods further include contacting the control sample with a compound or agent capable of detecting alpha-methylacyl-CoA racemase protein, and comparing the presence of alpha-methylacyl-CoA racemase protein in the control sample with the presence of alpha-methylacyl-CoA racemase protein in the test sample.

The invention also includes kits for detecting the presence of alpha-methylacyl-CoA racemase in a biological sample. For example, the kit can include a compound or agent capable of detecting alpha-methylacyl-CoA racemase protein or mRNA in a biological sample, and a standard. The compound or agent can be packaged in a suitable container. The kit can further comprise instructions for using the kit to detect alpha-methylacyl-CoA racemase protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably-labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein-stabilizing agent. The kit can also includes components necessary for detecting the detectable agent (e.g., an enzyme or a substrate). The kit can also contain a control sample or a series of control samples that can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed, aberrant or unwanted alpha-methylacyl-CoA racemase expression or activity.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted alpha-methylacyl-CoA racemase expression or activity. For example, such methods can be used to determine whether a subject can be effectively treated with an agent that modulates alpha-methylacyl-CoA racemase expression or activity.

The methods of the invention can also be used to detect genetic alterations in an alpha-methylacyl-CoA racemase gene, thereby determining if a subject with the altered gene is at risk for a disorder characterized by misregulation in alpha-methylacyl-CoA racemase protein activity or nucleic acid expression, such as a disorder associated with hematopoiesis or an immune disorder. In preferred embodiments, the methods include detecting, in a sample from the subject, the presence or absence of a genetic alteration characterized by at least one of an alteration affecting the integrity of a gene encoding an alpha-methylacyl-CoA racemase protein, or the misexpression of the alpha-methylacyl-CoA racemase gene. For example, such genetic alterations can be detected by ascertaining the existence of at least one of 1) a deletion of one or more nucleotides from an alpha-methylacyl-CoA racemase gene; 2) an addition of one or more nucleotides to a alpha-methylacyl-CoA racemase gene; 3) a substitution of one or more nucleotides of an alpha-methylacyl-CoA racemase gene, 4) a chromosomal rearrangement of an alpha-methylacyl-CoA racemase gene; 5) an alteration in the level of a messenger RNA transcript of an alpha-methylacyl-CoA racemase gene, 6) aberrant modification of an alpha-methylacyl-CoA racemase gene, such as of the methylation pattern of the genomic DNA, 7) the presence of a non-wild-type splicing pattern of a messenger RNA transcript of an alpha-methylacyl-CoA racemase gene, 8) a non-wild-type level of an alpha-methylacyl-CoA racemase protein, 9) allelic loss of an alpha-methylacyl-CoA racemase gene, and 10) inappropriate post-translational modification of an alpha-methylacyl-CoA racemase protein.

An alteration can be detected without a probe/primer in a polymerase chain reaction, such as anchor PCR or RACE-PCR, or, alternatively, in a ligation chain reaction (LCR), the latter of which can be particularly useful for detecting point mutations in the alpha-methylacyl-CoA racemase gene. This method can include the steps of collecting a sample of cells from a subject, isolating nucleic acid (e.g., genomic, mRNA or both) from the sample, contacting the nucleic acid sample with one or more primers which specifically hybridize to a alpha-methylacyl-CoA racemase gene under conditions such that hybridization and amplification of the alpha-methylacyl-CoA racemase gene occurs (if present), and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., (1990) Proc. Natl. Acad. Sci. USA 87:1874-1878), a transcriptional amplification system (Kwoh et al., (1989) Proc. Natl. Acad. Sci. USA 86:1173-1177), Q-Beta Replicase (Lizardi et al. (1988) Bio-Technology 6:1197), or other nucleic acid amplification methods, followed by the detection of the amplified molecules using techniques known to those of skill in the art.

In another embodiment, mutations in an alpha-methylacyl-CoA racemase gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis, and compared. Differences in fragment length sizes between sample and control DNA indicates that there are mutations in the sample DNA. Moreover, sequence specific ribozymes (see, for example, U.S. Pat. No. 5,498,531) can be used to score for the presence of specific mutations by development or loss of a ribozyme cleavage site.

In other embodiments, genetic mutations in alpha-methylacyl-CoA racemase can be identified by hybridizing a sample to control nucleic acids, e.g., DNA or RNA, by, e.g., two-dimensional arrays, or, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotide probes (Cronin et al. (1996) Human Mutation 7:244-255; Kozal et al. (1996) Nature Medicine 2:753-759). For example, genetic mutations in alpha-methylacyl-CoA racemase can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence an alpha-methylacyl-CoA racemase gene and detect mutations by comparing the sequence of the sample alpha-methylacyl-CoA racemase with the corresponding wild-type (control) sequence. Automated sequencing procedures can be utilized when performing the diagnostic assays ((1995) Biotechniques 19:448), including sequencing by mass spectrometry.

Other methods for detecting mutations in an alpha-methylacyl-CoA racemase gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad. Sci. USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in alpha-methylacyl-CoA racemase cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in alpha-methylacyl-CoA racemase genes. For example, single strand conformation polymorphism (SSCP) may be used to detect differences in electrophoretic mobility between mutant and wild-type nucleic acids (Orita et al. (1989) Proc. Natl. Acad. Sci. USA 86:2766, see also Cotton (1993) Mutat. Res. 285:125-144; and Hayashi (1992) Genet. Anal. Tech. Appl. 9:73-79). Single-stranded DNA fragments of sample and control alpha-methylacyl-CoA racemase nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci. USA 86:6230).

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell. Probes 6: 1). It is anticipated that in certain embodiments, amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an alpha-methylacyl-CoA racemase gene.

### Use of Alpha-Methylacyl-CoA Racemase Molecules as Surrogate Markers

The alpha-methylacyl-CoA racemase molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the alpha-methylacyl-CoA racemase molecules of the invention may be detected, and may be correlated with one or more biological states in vivo. For example, the alpha-methylacyl-CoA racemase molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached. Examples of the use of surrogate markers in the art include: Koomen et al. (2000) J. Mass. Spectrom. 35:258-264; and James (1994) AIDS Treatment News Archive 209.

The alpha-methylacyl-CoA racemase molecules of the invention may also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., an alpha-methylacyl-CoA racemase marker) transcription or expression, the amplified marker may be in a quantity which is more readily detectable than the drug itself. Also, the marker may be more easily detected due to the nature of the marker itself; for example, using the methods described herein, anti-alpha-methylacyl-CoA racemase antibodies may be employed in an immune-based detection system for an alpha-methylacyl-CoA racemase protein marker, or alpha-methylacyl-CoA racemase-specific radiolabeled probes may be used to detect an alpha-methylacyl-CoA racemase mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The alpha-methylacyl-CoA racemase molecules of the invention are also useful as pharmacogenomic markers. As used herein, a “pharmacogenomic marker” is an objective biochemical marker that correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., alpha-methylacyl-CoA racemase protein or RNA) for specific tumor markers in a subject, a drug or course of treatment may be selected that is optimized for the treatment of the specific tumor likely to be present in the subject. Similarly, the presence or absence of a specific sequence mutation in alpha-methylacyl-CoA racemase DNA may correlate with alpha-methylacyl-CoA racemase drug response. The use of pharmacogenomic markers therefore permits the application of the most appropriate treatment for each subject before administering the therapy.

### Pharmaceutical Compositions

The nucleic acid and polypeptides, fragments thereof, as well as anti-alpha-methylacyl-CoA racemase antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, that are compatible with pharmaceutical administration. Supplementary active compounds can also be incorporated into the compositions.

A pharmaceutical composition is formulated to be compatible with its intended route of administration. Examples of routes of administration include oral and parenteral, e.g., intravenous, intradermal, subcutaneous, inhalation, transdermal (topical), transmucosal, and rectal administration. Solutions or suspensions used for parenteral, e.g., intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including an agent in the composition that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents and/or adjuvant materials can be included as part of the composition. The tablets, pills, capsules, troches and the like can contain any of the following ingredients, or compounds of a similar nature: a binder, such as microcrystalline cellulose, gum tragacanth or gelatin; an excipient, such as starch or lactose; a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells using monoclonal antibodies directed towards viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for the lipidation of antibodies is described by Cruikshank et al. ((1997) J. Acquired Immune Deficiency Syndromes and Human Retrovirology 14:193).

The present invention encompasses agents that modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including hetero-organic and organo-metallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, and the drug moiety is not to be construed as limited to classical chemical therapeutic agents. For example, the drug moiety may be a protein or polypeptide possessing a desired biological activity. Such proteins may include, for example, a toxin such as abrin, ricin A, gelonin, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Methods of Treatment

The present invention provides for both prophylactic and therapeutic methods of treating a subject at risk of (or susceptible to) a disorder or having a disorder associated with aberrant or unwanted alpha-methylacyl-CoA racemase expression or activity, e.g., a cancer, particularly a metastasis of a prostate cancer, by administering a compound which decreases the expression or activity of alpha-methylacyl-CoA racemase. With regards to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype” or “drug response genotype”). Thus, another aspect of the invention provides methods for tailoring an individual's prophylactic or therapeutic treatment with either an alpha-methylacyl-CoA racemase molecule of the present invention or alpha-methylacyl-CoA racemase modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing a disease or condition (e.g., a cancer) in a subject associated with an aberrant or unwanted alpha-methylacyl-CoA racemase expression or activity, by administering to the subject an alpha-methylacyl-CoA racemase or an agent which modulates alpha-methylacyl-CoA racemase expression, or at least one alpha-methylacyl-CoA racemase activity. Subjects at risk for a disease caused or contributed to by aberrant or unwanted alpha-methylacyl-CoA racemase expression or activity can be identified by, for example, any or a combination of diagnostic or prognostic assays as described herein. Administration of a prophylactic agent can occur prior to the manifestation of symptoms characteristic of the alpha-methylacyl-CoA racemase aberrance, such that a disease or disorder is prevented or, alternatively, delayed in its progression.

As discussed, successful treatment of alpha-methylacyl-CoA racemase disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of alpha-methylacyl-CoA racemase disorders, e.g., certain cancers. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by alpha-methylacyl-CoA racemase expression is through the use of aptamer molecules specific for alpha-methylacyl-CoA racemase protein. Aptamers are nucleic acid molecules having a tertiary structure that permits them to specifically bind to protein ligands (see, e.g., Osborne et al. (1997) Curr. Opin. Chem. Biol. 1:5-9; and Patel (1997) Curr Opin Chem Biol 1:32-46). Since nucleic acid molecules may in many cases be more conveniently introduced into target cells than therapeutic protein molecules may be, aptamers offer a method by which alpha-methylacyl-CoA racemase protein activity may be specifically decreased without the introduction of drugs or other molecules which may have pluripotent effects.

Antibodies can be generated that are both specific for target gene product and that reduce target gene product activity. Such antibodies may, therefore, be administered in instances whereby negative modulatory techniques are appropriate for the treatment of alpha-methylacyl-CoA racemase disorders. For a description of antibodies, see the Antibody section above.

In circumstances wherein injection of an animal or a human subject with an alpha-methylacyl-CoA racemase protein or epitope for stimulating antibody production is harmful to the subject, it is possible to generate an immune response against alpha-methylacyl-CoA racemase through the use of anti-idiotypic antibodies (see, for example, Herlyn (1999) Ann. Med. 31:66-78; and Bhattacharya-Chatterjee and Foon (1998) Cancer Treat. Res. 94:51-68). If an anti-idiotypic antibody is introduced into a mammal or human subject, it should stimulate the production of anti-anti-idiotypic antibodies that are specific to the alpha-methylacyl-CoA racemase protein. Vaccines directed to a disease characterized by alpha-methylacyl-CoA racemase expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate disorders associated with alpha-methylacyl-CoA racemase expression or activity. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate alpha-methylacyl-CoA racemase activity is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be found in Ansell et al. (1996) Current Opinion in Biotechnology 7:89-94 and in Shea, (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be found in Vlatakis et al. (1993) Nature 361:645-647. Through the use of isotope-labeling, the “free” concentration of compound which modulates the expression or activity of alpha-methylacyl-CoA racemase can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al. (1995) Analytical Chemistry 67:2142-2144.

Another aspect of the invention pertains to methods of modulating alpha-methylacyl-CoA racemase expression or activity for therapeutic purposes. Accordingly, in an exemplary embodiment, the modulatory method of the invention involves contacting a cell with an alpha-methylacyl-CoA racemase or agent that modulates one or more of the activities of alpha-methylacyl-CoA racemase protein activity associated with the cell. An agent that modulates alpha-methylacyl-CoA racemase protein activity can be an agent as described herein, such as a nucleic acid or a protein, a naturally-occurring target molecule of a alpha-methylacyl-CoA racemase protein (e.g., a alpha-methylacyl-CoA racemase substrate or receptor), a alpha-methylacyl-CoA racemase antibody, a alpha-methylacyl-CoA racemase agonist or antagonist, a peptidomimetic of a alpha-methylacyl-CoA racemase agonist or antagonist, or other small molecule.

Examples of inhibitory agents include antisense alpha-methylacyl-CoA racemase nucleic acid molecules, anti-alpha-methylacyl-CoA racemase antibodies, and alpha-methylacyl-CoA racemase inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an alpha-methylacyl-CoA racemase protein or nucleic acid molecule. In one embodiment, the method involves administering an agent (e.g., an agent identified by a screening assay described herein), or combination of agents that modulates (e.g., upregulates or downregulates) alpha-methylacyl-CoA racemase expression or activity. In another embodiment, the method involves administering an alpha-methylacyl-CoA racemase protein or nucleic acid molecule as therapy to compensate for reduced, aberrant, or unwanted alpha-methylacyl-CoA racemase expression or activity.

Likewise, inhibition of alpha-methylacyl-CoA racemase activity is desirable in situations in which alpha-methylacyl-CoA racemase is abnormally upregulated and/or in which decreased alpha-methylacyl-CoA racemase activity is likely to have a beneficial effect.

### Pharmacogenomics

Agents or modulators that have an inhibitory effect on alpha-methylacyl-CoA racemase activity or expression as identified by a screening assay described herein can be administered to individuals to treat (prophylactically or therapeutically) alpha-methylacyl-CoA racemase-associated disorders associated with aberrant or unwanted alpha-methylacyl-CoA racemase activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an alpha-methylacyl-CoA racemase molecule or alpha-methylacyl-CoA racemase modulator as well as tailoring the dosage and/or therapeutic regimen of treatment with a alpha-methylacyl-CoA racemase molecule or alpha-methylacyl-CoA racemase modulator.

Pharmacogenomics deals with clinically significant hereditary variations in the response to drugs due to altered drug disposition and abnormal action in affected persons (see, for example, Eichelbaum et al. (1996) Clin. Exp. Pharmacol. Physiol. 23:983-985 and Linder et al. (1997) Clin Chem 43:254-266). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high-resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug's target is known (e.g., an alpha-methylacyl-CoA racemase protein), all common variants (e.g., alleles) of that gene can be fairly easily identified in the population and it can be determined if having one version of the gene versus another is associated with a particular drug response.

Alternatively, a method termed “gene expression profiling” can be utilized to identify genes that predict drug response. For example, the gene expression of an animal dosed with a drug (e.g., a alpha-methylacyl-CoA racemase molecule or alpha-methylacyl-CoA racemase modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a alpha-methylacyl-CoA racemase molecule or alpha-methylacyl-CoA racemase modulator, such as a modulator identified by one of the exemplary screening assays described herein.

The present invention further provides methods for identifying new agents, or combinations, that are based on identifying agents that modulate the activity of one or more of the gene products encoded by an alpha-methylacyl-CoA racemase gene, wherein these products may be associated with resistance of cells to a therapeutic agent. Specifically, the activity of alpha-methylacyl-CoA racemase can be used as a basis for identifying agents for overcoming agent resistance.

Monitoring the influence of agents (e.g., drugs) on the expression or activity of an alpha-methylacyl-CoA racemase protein can be applied in clinical trials. For example, the effectiveness of an agent determined by a screening assay to decrease alpha-methylacyl-CoA racemase gene expression, protein levels, or down-regulate alpha-methylacyl-CoA racemase activity, can be monitored in clinical trials of subjects exhibiting increased alpha-methylacyl-CoA racemase gene expression, protein levels, or upregulated alpha-methylacyl-CoA racemase activity. In such clinical trials, the expression or activity of an alpha-methylacyl-CoA racemase gene, and preferably, other genes that have been implicated in, for example, a alpha-methylacyl-CoA racemase-associated disorder can be used as a “read out” or markers of the phenotype of a particular cell.

The contents of all references, patents and published patent applications cited throughout this application are incorporated herein by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for determining whether a patient has prostate cancer, comprising:
(a) obtaining a test sample from the patient;
(b) measuring the expression of alpha-methylacyl-CoA racemase in the test sample;
(c) determining that the patient has prostate cancer if the expression of alpha-methylacyl-CoA racemase in the test sample is greater than a predetermined value.

2. The method of claim 1, wherein the method determines whether the prostate cancer is metastatic prostate cancer.

3. The method of claim 1, wherein the test sample comprises cells from a tissue selected from the group consisting of prostate, liver and lymph node.

4. The method of claim 1, wherein the expression of alpha-methylacyl-CoA racemase is determined by measuring the amount of activity of alpha-methylacyl-CoA racemase in the test sample.

5. The method of claim 1, wherein the expression of alpha-methylacyl-CoA racemase is determined by measuring the amount of alpha-methylacyl-CoA racemase nucleic acid in the test sample.

6. The method of claim 1, wherein the expression of alpha-methylacyl-CoA racemase is determined by measuring the amount of alpha-methylacyl-CoA racemase polypeptide in the test sample.

7. The method of claim 5, wherein the alpha-methylacyl-CoA racemase nucleic acid comprises a nucleotide sequence selected from the group consisting of SEQ ID NO:1, SEQ ID NO:3, SEQ ID NO:4, SEQ ID NO:6, SEQ ID NO:8, SEQ ID NO:10 and the complement of any of the foregoing.

8. The method of claim 5, wherein the amount of nucleic acid encoding alpha-methylacyl-CoA racemase in the test sample is measured in a method which comprises hybridizing a nucleic acid molecule in the test sample under stringent conditions to an alpha-methylacyl-CoA racemase nucleic acid.

9. The method of claim 6, wherein the alpha-methylacyl-CoA racemase polypeptide comprises an amino acid sequence selected from the group consisting of SEQ ID NO:2, SEQ ID NO:5, SEQ ID NO:7, SEQ ID NO:9, SEQ ID NO:11, and any of the foregoing with conservative amino acid substitutions.

10. The method of claim 6, wherein the amount of alpha-methylacyl-CoA racemase polypeptide in the test sample is measured in a method which comprises exposing the test sample to an antibody that selectively binds to the alpha-methylacyl-CoA racemase polypeptide.

11. A method for selecting a patient for therapy with a compound which decreases alpha-methylacyl-CoA racemase expression, the method comprising:
(a) obtaining a test sample from the patient;
(b) determining the expression of alpha-methylacyl-CoA racemase in the test sample;
(c) comparing the amount of expression of alpha-methylacyl-CoA racemase in the test sample to a predetermined value; and
(d) selecting the patient for therapy with a compound which decreases alpha-methylacyl-CoA racemase expression when the amount of alpha-methylacyl-CoA racemase expression in the test sample is greater than the predetermined value.

12. The method of claim 11, wherein the test sample comprises cells from a tissue selected from the group consisting of prostate, liver and lymph node.

13. The method of claim 11, wherein the expression of alpha-methylacyl-CoA racemase is determined by measuring the amount of alpha-methylacyl-CoA racemase nucleic acid in the test sample.

14. The method of claim 11, wherein the expression of alpha-methylacyl-CoA racemase is determined by measuring the amount of alpha-methylacyl-CoA racemase polypeptide in the test sample.

15. A method for identifying candidate therapeutic agents for the treatment of prostate cancer, the method comprising:
(a) obtaining a test sample comprising prostate tumor cells;
(b) exposing the test sample to a test compound;
(c) measuring the level of expression of alpha-methylacyl-CoA racemase in the test sample exposed to the test compound;
(d) determining that the test compound is a candidate therapeutic agent for the treatment of prostate cancer if the level of expression of alpha-methylacyl-CoA racemase in the test sample exposed to the test compound is less than a predetermined value.

16. A method for determining whether a therapeutic treatment for prostate cancer should be continued, the method comprising:
(a) obtaining a first test sample from a prostate cancer patient at a first time;
(b) obtaining a second test sample from the patient at a second, later time;
(c) measuring the expression of alpha-methylacyl-CoA racemase in the first and second test samples; and
(d) determining that the therapeutic treatment should be continued when the expression of alpha-methylacyl-CoA racemase in the second test sample is less than or equal to the expression of alpha-methylacyl-CoA racemase than in the first test sample.

17. An isolated nucleic acid molecule comprising a nucleotide sequence selected from the group consisting of:
(a) SEQ ID NO:1, SEQ ID NO:3, SEQ ID NO:4, SEQ ID NO:6, SEQ ID NO:8, SEQ ID NO:10, or the complement of any of the foregoing; and
(b) a sequence that encodes a polypeptide comprising an amino acid sequence selected from the group consisting of SEQ ID NO:2, SEQ ID NO:5, SEQ ID NO:7, SEQ ID NO:9, SEQ ID NO:11, or any of the foregoing with conservative amino acid substitutions.

18. An isolated polypeptide comprising an amino acid sequence selected from the group consisting of SEQ ID NO:2, SEQ ID NO:5, SEQ ID NO:7, SEQ ID NO:9, SEQ ID NO:11, and any of the foregoing with conservative amino acid substitutions.
